(19) United States
(12) Patent Application Publication
(10) Pub. No.: US 2024/0000811 A1
(43) Pub. Date: Jan. 4, 2024

(54) INOSITOL HEXAKISPHOSPHATE ANALOGUES FOR TREATMENT OF CALCIFICATION ASSOCIATED KIDNEY DISEASES

(71) Applicants: INOSITEC AG, Zurich (CH); ETH ZÜRICH, Zürich (CH)

(72) Inventors: Mattias Emanuel IVARSSON, Zürich (CH); Jean-Christophe LEROUX, Zürich (CH); Anna KLETZMAYR, Zürich (CH); Makoto KURO-O, Saitama (JP); Hirosaka HAYASHI, Shimotsuke (JP)

(73) Assignees: VIFOR (INTERNATIONAL) AG, St. Gallen (CH); ETH ZÜRICH, Zürich (CH)

(21) Appl. No.: 18/253,601

(22) PCT Filed: Nov. 19, 2021
(86) PCT No.: PCT/EP2021/082374

§ 371 Date: May 19, 2023

(30) Foreign Application Priority Data
Nov. 20, 2020 (EP) 20209090.8
Nov. 20, 2020 (EP) 202090908

(51) Int. Cl. A61K 31/6615; A61P 3/14; A61P 13/12

(52) U.S. Cl. CPC A61K 31/6615; A61P 3/14; A61P 13/12

(57) ABSTRACT

The invention is related to an inositol polyphosphate oligo alkyl ether compound, or its pharmaceutically acceptable salt, for use in treatment or prevention of a disease associated with formation of calcium salt crystals.

The present invention relates to compounds and compositions for use in treatment of conditions associated with calcification of tissue, particularly kidney tissue, caused by deposition of, or exposure to, calcium phosphate (CaP) and other calcium precipitates.

### BACKGROUND OF THE INVENTION

WO2013045107 (A1) first discloses the concept of using inositol polyphosphate polyalkylether derivatives as pharmaceutical agents. Initially conceived as an agent of considerable potential to neutralize C. difficile toxin in the colon lumen, subsequent analysis found that the compounds are highly effective in reducing calcification when applied systemically, as first disclosed in WO2017098047 (A1), U.S. Ser. No. 10/624,909 (B2) and US20200247837 (A1).

WO2020058321 (A1) discloses further compounds based on inositol polyphosphate scaffolds with improved pharmacological properties.

All patent documents mentioned in the present specification are incorporated herein by reference in their entirety.

The objective of the present invention is to provide further advantageous applications of inositol polyphosphate polyalkylether derivatives. This objective is attained by the subject-matter of the independent claims of the present specification.

### SUMMARY OF THE INVENTION

In one aspect, the invention provides an inositol polyphosphate oligo alkyl ether compound, or its pharmaceutically acceptable salt, for use in treatment or prevention of a disease associated with formation of calcium salt precipitate or calcium salt crystals. In the broadest sense, the disease addressed by the compounds of the invention, is chronic kidney disease associated with calcium salt precipitation, particularly with the formation of precipitates comprised of calcium phosphate and/or oxalate.

Particular diseases the treatment of which is likely to benefit from administration of the compounds described in here include renal fibrosis, particularly when associated with calcification, or exposure to calcium phosphate or calcium oxalate precipitates, of renal tissue, renal inflammation, particularly when associated with calcification or exposure to calcium phosphate or calcium oxalate precipitates of renal tissue, nephritis, particularly interstitial nephritis, glomerulonephritis, phosphate-induced renal fibrosis, phosphate-induced chronic kidney disease, chronic kidney disease associated with hyperphosphatemia, progression of chronic kidney disease, phosphate toxicity, hyperphosphaturia, hyperphosphatemia, and/or hyper-FGF23-emia.

According to an alternative of this aspect of the invention, an inositol polyphosphate oligo alkyl ether compound, or its pharmaceutically acceptable salt, is provided for use in treatment or prevention of a disease associated with formation of calcium salt precipitate or calcium salt crystals, the disease being selected from vascular calcification, coronary artery disease, vascular stiffening, valvular calcification, nephrocalcinosis, calcinosis cutis, kidney stones, and chondrocalcinosis.

One of the key findings emanating from the data presented here, which the inventors believe is truly novel, is that a protective effect was observed in vivo even in the absence of overt calcifications. The mouse model used in the examples is one where, in the time frame tested, CaP precipitates form in the renal tubules but it is not actually possible to detect calcification of the kidney per se by histology (von Kossa staining is negative). Calcification does appear if one maintains the animals for longer or if one performs nephrectomy at the start of the experiment to accelerate the process under the conditions employed in the absence of treatment with inhibitor. A protective effect is of course also be expected when calcifications are present and measurable (which is the case in the cell assay used in the earlier examples).

### Terms and Definitions

For purposes of interpreting this specification, the following definitions will apply and whenever appropriate, terms used in the singular will also include the plural and vice versa. In the event that any definition set forth below conflicts with any document incorporated herein by reference, the definition set forth shall control.

The terms “comprising,” “having,” “containing,” and “including,” and other similar forms, and grammatical equivalents thereof, as used herein, are intended to be equivalent in meaning and to be open ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of.”

Where a range of values is provided, it is understood that each intervening value, to the tenth of the unit of the lower limit, unless the context clearly dictate otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

Reference to “about” a value or parameter herein includes (and describes) variations that are directed to that value or parameter per se. For example, description referring to “about X” includes description of “X.”

As used herein, including in the appended claims, the singular forms “a,” “or,” and “the” include plural referents unless the context clearly dictates otherwise.

Unless defined otherwise, all technical and scientific terms used herein have the same meaning as commonly understood by one of ordinary skill in the art (e.g., in cell culture, molecular genetics, nucleic acid chemistry, hybridization techniques and biochemistry). Standard techniques are used for molecular, genetic and biochemical methods (see generally, Sambrook et al., Molecular Cloning: A Laboratory Manual, 4th ed. (2012) Cold Spring Harbor Laboratory Press, Cold Spring Harbor, N.Y. and Ausubel et al., Short Protocols in Molecular Biology (2002) 5th Ed, John Wiley & Sons, Inc.) and chemical methods.

In the context of the present specification, the term oligo-alkylether relates to oligo-ethylene glycol and close chemical relatives such as oligo-propylene glycol and oligo-glycerol. The term “oligo” signifies that more than one, particularly from 2 to 20, more particularly from 2 to 12 monomers (—CH₂—CH₂—O—) in the case of oligo-ethylene glycol, (—CH(CH₃)—CH₂—O—) in the case of oligo-propylene glycol) are present.

In the context of the present specification, the term inositolpolyphosphate relates to cyclohexane-hexol (inositol, cyclohexane-1,2,3,4,5,6-hexol) wherein each OH is substituted by a phosphate ester moiety unless the OH is substituted by an oligo-alkylether moiety according to the preceding definition. In particular embodiments, the inositol scaffold is myo-inositol ((1R,2S,3R,4R,5S,6S)-cyclohexane-1,2,3,4,5,6-hexol).

The term inositol polyphosphate oligo-alkylether compound in the context of the present specification relates to a compound comprising one or several inositol polyphosphate moieties as defined above, and at least one oligoalkylether.

The term calcification in the context of the present specification relates to the formation of calcium precipitates in the affected tissue, particularly renal tissue.

### DETAILED DESCRIPTION OF THE INVENTION

A first aspect of the invention relates to an inositol polyphosphate oligo alkyl ether compound, or its pharmaceutically acceptable salt, for use in treatment or prevention of a disease associated with formation of calcium salt precipitate or calcium salt crystals. In the broadest sense, the disease is chronic kidney disease associated with calcium salt precipitation, particularly with the formation of precipitates comprised of calcium phosphate and/or oxalate.

Without wanting to be limited by theory, the inventors draw the conclusion from that results provided herein, that pathophysiologic mechanisms of the diseases mentioned here involve, as a first step, precipitation of calcium phosphate matter, which subsequently grows, adheres to cells (and perhaps grows further once adhered to cells). The interaction of the calcium phosphate precipitates with renal tubular cells, whether the precipitates are large or small, causes damage to the cells.

The compounds and compositions provided herein apparently stop new precipitates from forming/growing but it also stop existing precipitates from adhering to the cells, both of which confer a protective effect. There is therefore a dual mode of action.

Calcium Precipitates

The chemical composition of calcium crystalline precipitates associated with renal and cardiovascular disease is well studied (see Elliot, J Urology 100 (1968), 687-693; Xie et al., Cryst Growth Des. 2015 Jan. 7; 15(1): 204-211). In principle, the treatment according to the invention is capable of preventing or ameliorating any condition in which deposition of calcium phosphate, oxalate and mixed calcium phosphate-oxalate crystals play a role.

Calcium phosphate is found in different crystal forms in pathologic deposits in the human body; these include hydroxyapatite (hydroxylapatite, HA, Ca₁₀(PO₄)₆(OH)₂), brushite (CaHPO₄*2H₂O), monetite, and various amorphous calcium phosphate salts.

Calcium oxalate in pure form is found as a mono- (whewellite), di- (weddellite) and tri-hydrate, and often associated with other deposits, mainly phosphate salts.

The results presented in the examples show that any of the cellular sequelae of calcium salt crystal formation, even when associated with deposits preceding the stage of macroscopically detectable calcification, can be prevented by treatment according to the invention as described in the present specification.

Diseases Associated to Calcium Precipitation that Benefit from Treatment According to the Invention

“Renal fibrosis” or “tubulointerstitial fibrosis” associated with formation of, and/or tissue exposure to, calcium salt crystals, refers to a thickening and scarring of kidney tissue, due to unsuccessful wound-healing after chronic injury due to exposure to calcium phosphate, calcium oxalate or mixed CaP/CaOx precipitates. During this process fibrotic matrix deposition continues unchecked leading to glomerulosclerosis, tubular atrophy and interstitial fibrosis. Patients suffering from said disease can experience intense abdominal pain (with bleeding or haemorrhaging), swelling and discoloration in one or both legs, and ultimately progress to chronic kidney disease.

“Renal inflammation” or “nephritis” is defined as a complex network of interactions between renal parenchymal cells and resident immune cells, such as macrophages and dendritic cells, coupled with recruitment of circulating monocytes, lymphocytes, and neutrophils. Once stimulated, these cells activate specialized structures such as Toll-like receptor and Nod-like receptor (NLR). By detecting danger-associated molecules, these receptors can set in motion major innate immunity pathways such as nuclear factor κB (NF-κB) and NLRP3 inflammasome, causing metabolic reprogramming and phenotype changes of immune and parenchymal cells and triggering the secretion of a number of inflammatory mediators that can cause irreversible tissue damage and functional loss. In CKD, a chronic inflammation leads to a progressively decreasing glomerular filtration rate (GFR) that can ultimately result in kidney failure (end stage renal disease, ESRD).

To the extent that renal inflammation/nephritis is associated or caused by interaction of renal tissue with calcium deposits, the treatment according to the invention is expected to ameliorate or prevent the condition. The examples shown herein demonstrate that calcium phosphate deposition/exposure leads to upregulation of inflammation and fibrosis markers in the kidneys, therefore any disease that consists of inflammation and fibrosis of the kidneys should benefit from treatment that inhibits the formation of deposits. For each of these inflammatory conditions, there may also be other, even concurrent causes for the inflammation and fibrosis, as both of these are often multifactorial processes. Abrogation of one contributing factor, however, is expected to improve the overall clinical picture.

“Glomerulonephritis” refers to a group of diseases that are characterized by inflammatory changes in glomerular capillaries. Patients suffering from said group of diseases can experience proteinuria, impaired renal function in some cases paired with fluid retention, hypertension and oedema. Glomerulonephritis can occur as a primarily renal disease as well as indicate a systematic disease process. When associated with formation of, and/or tissue exposure to, calcium salt crystals, glomerulonephritis is expected to benefit from treatment with the compounds described herein.

“Interstitial nephritis” refers to inflammation of the renal interstitium which can be caused by unbalanced levels of calcium. Symptoms include increased urine output, hematuria, changes in mental status, swelling. This condition, if associated with formation of, and/or tissue exposure to, calcium salt crystals, is expected to benefit from treatment with the compounds described herein.

“Phosphate-induced renal fibrosis” may be associated with a range of calcium and phosphate concentrations that lead to precipitation in the renal tubules, see the data presented in FIG. 2: i.e. calcium>2 or 5 mmol/L and phosphate>5 or 7 mmol/L. Of note, there can be elevated phosphate in renal tubular fluid (leading to its precipitation with calcium) even when detectable plasma phosphate levels are normal (i.e. in the absence of hyperphosphatemia). Elevated plasma phosphate is a terminal consequence of end-stage renal disease that occurs when kidney function is below a 20% threshold.

“Phosphate-induced chronic kidney disease” Phosphate-induced CKD can occur even if plasma phosphate levels are normal (i.e. in earlier stage CKD patients). Reference values can be calcium>2 or 5 mmol/L and phosphate>5 or 7 mmol/L in the renal tubules.

“Chronic kidney disease associated with hyperphosphatemia” refers to hyperphosphatemia occurring in progressive CKD, due to the increasing loss of GFR. This leads to the blocking of tubular phosphate reabsorption and therefore increased phosphate retention, in other words, less phosphate clearance, impairing phosphate homeostasis (Sharon M. Moe, Prim Care. 2008 June; 35(2): 215-vi.). A useful reference value to characterize patients who are expected to benefit from the treatment according to the invention can be a plasma phosphate level of >1.46 mmol/Ls.

“Progression of chronic kidney disease” refers to five stages, ranging from the first stage (mild damage, eGFR 90 or greater) to the fifth (complete kidney failure, eGFR less than 15). Current guidelines determine the critical GFR for CKD to be less than 60 mL/min per 1.73 m² over a period of 3 months.

“Phosphate toxicity” refers to a dysregulated renal phosphate excretion and reabsorption, impairing phosphate homeostasis, which can cause severe damage of kidney tissue. (Razzague, Clin Sci (Lond). 2011 February; 120(3): 91-97). To the extent that this condition causes tissue damage associated with formation of, and/or tissue exposure to and/or deposition of crystals which can avoided by the treatment according to the invention, the treatment is indicated for patient suffering from the condition.

“Hyperphosphaturia” or “phosphaturia” refers to a high level of phosphate in urine. To the extent that this condition causes tissue damage associated with formation of, and/or tissue exposure to and/or deposition of crystals which can avoided by the treatment according to the invention, the treatment is indicated for patient suffering from the condition.

“Hyperphosphatemia” refers to an elevated (>4.5 mg/dL; >1.46 mmol/L) phosphate level in blood. To the extent that this condition causes tissue damage associated with formation of, and/or tissue exposure to and deposition of crystals which can avoided by the treatment according to the invention, the treatment is indicated for patient suffering from the condition.

“Hyper-FGF23-emia” refers to an increased fractional phosphate excretion, paired with a decrease of serum phosphate levels, due to elevated fibroblast growth factor (FGF)-23 levels.

“Vascular calcification” refers to the pathological deposition of minerals in the vascular system often observed in patients suffering from CDK or diabetes. The elevated calcium and/or phosphate levels can be the result of metabolic dysregulation caused by diabetes, dyslipidemia, oxidative stress, uremia, and hyperphosphatemia, which lead to osteoblast-like cell formation, appearance of calcified deposits and stiffening in the vessel wall.

“Coronary artery disease” or “artheroslerotic heart disease” refers to an accumulation of plaque in damaged inner layers of the coronary artery. Factors such as inflammatory cells, lipoproteins and calcium attach to the plaque leading to further stenosis. Progression of this disease can ultimately lead to myocardial infarction or stroke.

“Vascular stiffening” refers to stiffening of the arterial wall due to calcification. Vascular stiffening consists of lower elasticity of the vasculature leading to an increased pulse wave pressure.

“Valve calcification”, particularly aortic valve calcification, refers to an active dysregulation of normal homeostatic processes and hemodynamic changes, such as ECM degradation, fibrosis, lipid accumulation, and neo-angiogenesis of the valve tissue, concurrent with calcification of the valve, in particular the aortic and mitral valves.

“Nephrocalcinosis” refers to the deposition of calcium salts in the renal parenchyma, particularly in the medulla (medullary nephrocalcinosis) or cortex (cortical nephrocalcinosis) of the kidney.

“Calcinosis cutis” refers to the deposition of calcium phosphate precipitates within the skin, particularly within the extremities. If the solubility point of calcium and phosphate are exceeded, precipitation of calcium salts and deposition as amorphous hydroxyapatite occur.

“Kidney stones”, “renal calculi”, “nephrolithiasis”, and “urolithiasis” refer to a mineral deposit in renal tissue, which is due to the accumulation and therefore supersaturation of urine (hypercalcuria).

“Chondrocalcinosis” refers to the accumulation of calcium phosphate in joints.

Particular conditions the treatment of which is expected to benefit, based on the examples described in here, from administration of the compounds described in here further include aortic valve stenosis, peripheral artery disease and brain calcification.

The data shown in example 1 confirm the utility of the compounds disclosed herein as effective in reducing or inhibiting calcium phosphate crystal formation, in the prevention and treatment of idiopathic calcium nephrolithiasis/idiopathic calcium kidney stones, particularly those mainly consisting of calcium phosphate, calcium oxalate, or mixtures thereof.

Compounds Having One Inositol Scaffold

In certain embodiments, the inositol polyphosphate oligo alkyl ether compound, or its pharmaceutically acceptable salt, for use in treatment or prevention of the diseases laid out above, is described by a general formula I, wherein one or two or three X are oligo-ethylene glycol and the remaining X are OPO₃²⁻.

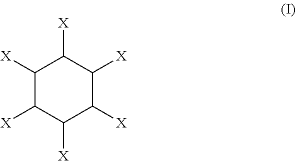

As shown in the examples provided herein, both mono- and bis-oligo-ethylene glycol derivatives of varying chain length confer inhibition of calcium precipitate formation in a cellular assay. The inositol scaffold can have any stereochemistry. The inventors worked preferably with myo-inositol.

When dissolved in aqueous medium at physiological pH, it is apparent to the skilled artisan that the compounds mentioned herein are anionic and will be accompanied by cations. The buffers used in the screens comprise mainly sodium (408 mmol/L), and traces of potassium (0.26 mmol/L) and magnesium (4 mmol/L).

In certain particular embodiments, two of the inositol substituents X shown in the above formula I are oligo-ethylene glycol and the remaining four X are OPO₃²⁻.

Examples for compounds that can be advantageously used in treatment according to the invention include general formulas I-1, I-2, I-3 and I-4, wherein in each case, X is phosphate and R¹ is an oligo-ethylene glycol as set forth herein:

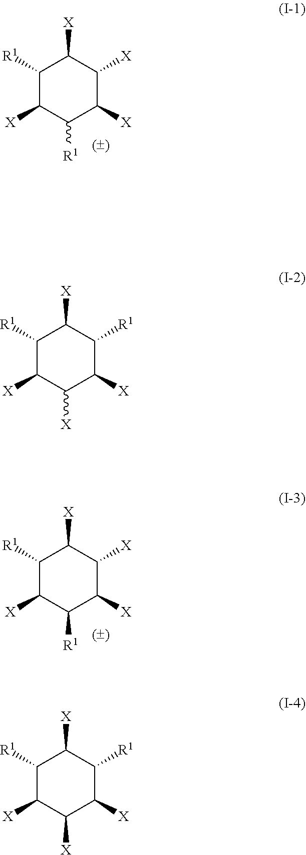

In one more particular embodiment thereof, the scaffold is myo-inositol and the oligo-ethylene glycol substituents are on position 4 and 6, with the rest of the substituents being phosphate. In an even more particular embodiment, the oligo-ethylene glycol substituents are O—(CH₂—CH₂O)₂—CH₃.

In other particular embodiments, one of the inositol substituents X shown in the above formula I is oligo-ethylene glycol and the remaining five X are OPO₃²⁻. In one more particular embodiment thereof, the scaffold is myo-inositol and the oligo-ethylene glycol substituent is on position 4 or 6, particularly on 6, with the rest of the substituents being phosphate. In an even more particular embodiment, the oligo-ethylene glycol substituent is O—(CH₂—CH₂O)₂—CH₃.

In yet other particular embodiments, three of the inositol substituents X shown in the above formula I are oligo-ethylene glycol and the remaining three X are OPO₃²⁻. Examples thereof include general formulas I-5 and I-6, wherein in each case, X is phosphate and R¹ is an oligo-ethylene glycol as set forth herein:

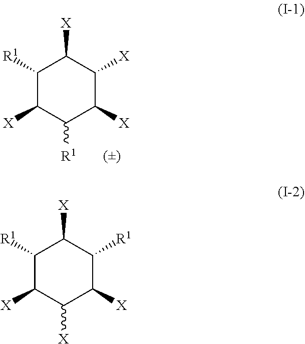

In certain embodiments, the oligo-ethylene glycol substituent (or substituents) of the inositol polyphosphate oligo alkyl ether compound, or its pharmaceutically acceptable salt, provided for use according to the invention herein, is described by a formula O—(CH₂—CH₂—O)ₙCH₃, with n being selected from an integer between 2 and 20, particularly n being 2 to 12. Different parameters of the compounds' physiological activity, pharmacological parameters and aspects of manufacture will influence which value of n is optimal.

In certain particular embodiments, the oligo-ethylene glycol substituent (or substituents) of the inositol polyphosphate oligo alkyl ether compound, or its pharmaceutically acceptable salt, provided for use according to the invention herein, is described by a formula O—(CH₂—CH₂—O)ₙCH₃, wherein n is 2. Table 1 of the examples shows the particular advantage of OEG₂-IP5 and (OEG₂)₂-IP4, both of which are characterized by n being 2.

Certain particular embodiments of the compound for use as specified herein are described by any one of the formulas:

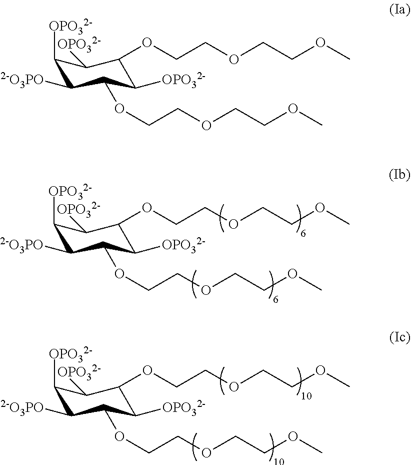

Certain particular embodiments of the compound for use as specified herein are described by any one of the formulas:

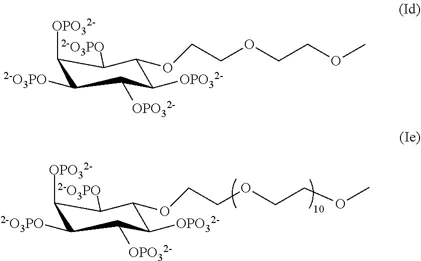

Other embodiments of compounds for use according to aspect of the invention include:

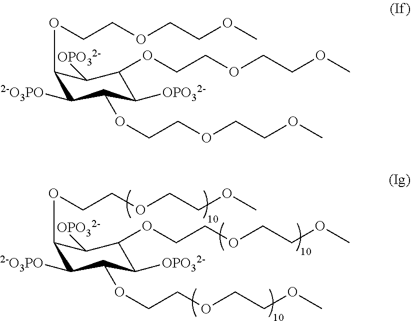

Compounds Having More than One Inositol Scaffold

In certain embodiments, the inositol polyphosphate oligo alkyl ether compound, or its pharmaceutically acceptable salt, for use in treatment or prevention of the diseases laid out above, is described by a general formula II, wherein each X is OPO₃²⁻ and L is —(O—CH₂—CH₂)ₘ—O— with m having a value between 5 and 15, particularly between 6 and 12.

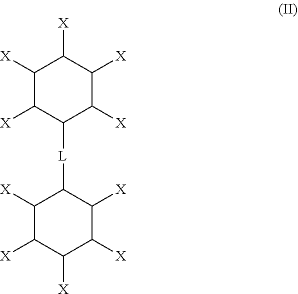

In one particular embodiment, m is 7. In one particular embodiment, m is 9. In one particular embodiment, m is 10.

In one especially particular embodiment, m is 8.

Certain particular embodiments of the compound for use as specified herein are described by any one of the formulas II-a (also referred to as OEG₄-(IP5)₂ herein) and II-b (also referred to as OEG₃-(IP5)₂ herein):

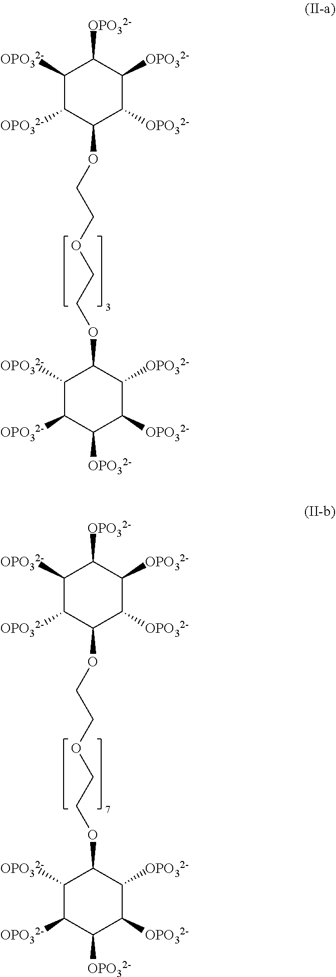

In certain particular embodiments, the binuclear inositol polyphosphate oligo-alkyl ether compound of general formula II, or its pharmaceutically acceptable salt, for use according to the invention, is characterized by both inositol moieties being myo-inositol.

Any of the inositol polyphosphate oligo-alkyl ether compounds, or its pharmaceutically acceptable salt, are provided for use in treatment or prevention of a disease associated with the formation of calcium phosphate salt or other solid precipitate in the body, particularly in renal tissue.

The administration of compounds having two oligo-ethylene glycol moieties attached to a myo-inositol tetrakisphosphate scaffold (I a) is of particular advantage in treatment or prevention of diseases as set forth herein that are associated with precipitation of calcium phosphate solid matter, and in prevention or treatment of mixed phosphate-oxalate calcium precipitates, or precipitates that mainly contain oxalate but originate from calcium phosphate nuclei, as evidenced by the data provided in example 1 and as exemplified by the growth of calcium oxalate kidney stones on calcium phosphate-based Randall's plaque.

As shown in the examples, the bipegylated compounds such as (OEG2)₂-IP4 also have a protective effect in the context of mixed precipitates (CaP+CaOx).

The administration of compounds having two myo-inositol pentakisphosphate scaffolds (II) linked by an oligo-ethylene glycol bridge, particularly one having eight —(O—CH₂—CH₂) repeats, is of particular advantage in treatment or prevention of diseases as set forth herein that are associated with precipitation of calcium oxalate solid matter.

The skilled person is aware that any specifically mentioned drug compound mentioned herein may be present as a pharmaceutically acceptable salt of said drug. Pharmaceutically acceptable salts comprise the ionized drug and an oppositely charged counterion. Non-limiting examples of pharmaceutically acceptable cationic salt forms include aluminium, benzathine, calcium, ethylene diamine, lysine, magnesium, meglumine, potassium, procaine, sodium, tromethamine and zinc.

Method of Manufacture and Method of Treatment According to the Invention

The invention further encompasses, as an additional aspect, the use of an inositol polyphosphate oligo alkyl ether compound, or its pharmaceutically acceptable salt, as specified in detail above, for use in a method of manufacture of a medicament for the treatment or prevention of a disease associated with formation of calcium salt precipitate or calcium salt crystals, specifically in a disease selected from renal fibrosis, particularly when associated with calcification of renal tissue, renal inflammation, particularly when associated with calcification of renal tissue, nephritis, particularly interstitial nephritis, glomerulonephritis, phosphate-induced renal fibrosis, phosphate-induced chronic kidney disease, chronic kidney disease associated with hyperphosphatemia, progression of chronic kidney disease, phosphate toxicity, hyperphosphaturia, hyperphosphatemia, and/or hyper-FGF23-emia.

The compounds of the invention are similarly provided for use in a method of manufacture of a medicament for the treatment or prevention of a disease associated with formation of calcium salt precipitate or calcium salt crystals, the disease being selected from vascular calcification, coronary artery disease, vascular stiffening, valvular calcification, nephrocalcinosis, calcinosis cutis, kidney stones, and chondrocalcinosis.

Similarly, the invention encompasses methods of treatment of a patient having been diagnosed with a disease associated with formation of calcium salt precipitate or calcium salt crystals, specifically in a disease selected from renal fibrosis, particularly when associated with calcification of renal tissue, renal inflammation, particularly when associated with calcification of renal tissue, nephritis, particularly interstitial nephritis, glomerulonephritis, phosphate-induced renal fibrosis, phosphate-induced chronic kidney disease, chronic kidney disease associated with hyperphosphatemia, progression of chronic kidney disease, phosphate toxicity, hyperphosphaturia, hyperphosphatemia, and/or hyper-FGF23-emia. This method entails administering to the patient an effective amount of an inositol polyphosphate oligo alkyl ether compound, or its pharmaceutically acceptable salt, as specified in detail herein.

Pharmaceutical Compositions and Administration

Another aspect of the invention relates to a pharmaceutical composition comprising a compound of the present invention, or a pharmaceutically acceptable salt thereof, and a pharmaceutically acceptable carrier. In further embodiments, the composition comprises at least two pharmaceutically acceptable carriers, such as those described herein.

In certain embodiments of the invention, the compound of the present invention is typically formulated into pharmaceutical dosage forms to provide an easily controllable dosage of the drug and to give the patient an elegant and easily handleable product.

In certain embodiments, the pharmaceutical composition for use according to the invention is formulated for administration by intradermal or subcutaneous injection.

The dosage regimen for the compounds of the present invention will vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient, and the effect desired. In certain embodiments, the compounds of the invention may be administered in a single daily dose, or the total daily dosage may be administered in divided doses of two, three, or four times daily.

The pharmaceutical composition for use according to the present invention can be subjected to conventional pharmaceutical operations such as sterilization and/or can contain conventional inert diluents or buffering agents, as well as adjuvants, such as preservatives, stabilizers, surfactants and buffers, etc. They may be produced by standard processes, for instance by conventional mixing, dissolving or lyophilizing processes. Many such procedures and methods for preparing pharmaceutical compositions are known in the art, see for example L. Lachman et al. The Theory and Practice of Industrial Pharmacy, 4th Ed, 2013 (ISBN 8123922892).

The invention is further illustrated by the following examples and figures, from which further embodiments and advantages can be drawn. These examples are meant to illustrate the invention but not to limit its scope.

### DESCRIPTION OF THE FIGURES

FIG. 1 shows an overview of the developed calcification profiling platform. (A) Outline of the workflow. (B) Overview of the output of the analytical pipeline. Example images of brightfield (column 1), CellMask (column 2), Hoechst (column 4) and calcein (column 6) stainings and two zoomed-in regions of interests (ROI) of RPTEC cells treated with 5/7 mM Ca/P are shown. CellMask and Hoechst channel images were used for single cell segmentation. Hoechst local maxima (indicated in dark blue, column 4) and the CellMask binary image served as seeds and input image, respectively, for the watershed algorithm. Comparison of the final cell segmentation in blue overlayed with the CellMask binary image in red is shown in column 3. The final segmentation and the Hoechst seeds are shown in column 5. Binary images of the calcein channel were generated by adaptive thresholding. Individual labelled CaP regions are shown in column 7.

FIG. 2 shows effects of increasing concentrations of Ca/P on RPTEC in vitro. (A) Heatmap and hierarchical clustering of the Ca/P treatment conditions and extracted image features. (B) Selected single features describing cellular changes upon increasing Ca/P concentrations are depicted. Total cell count, dead cell count, single cell area and single cell solidity, a measure of cell compactness, are shown. (C) Selected single features describing changes in the CaP deposition and membrane pattern upon increasing Ca/P concentrations are depicted. The total area of the binary calcein staining, colorimetric quantification of calcium content extracted from the monolayer, the maximum intensity of the calcein fluorescence, structural similarity index metric (SSIM) and the correlation of the CellMask channel image are shown. (D) Example brightfield, calcein, CellMask and EthD channel images and two zoomed-in regions of interests (ROI) of RPTEC treated with 2/5 mM Ca/P are represented. Mean scaled values per individual experiment are plotted in colored circles, except for total calcium quantification absolute values per individual experiment were used (N=3). Mean of three individual experiments and SD is plotted as grey horizontal and vertical line respectively, one-way ANOVA with Dunnet's multiple comparison between each concentration to 1/1 mM Ca/P was performed (* p<0.01).

FIG. 3 shows an overview of IP6 analogues tested in solution.

FIG. 4 shows inhibitory properties of (OEG₂)₂-IP4 on Ca/P induced changes of RPTEC in vitro. (A) Heatmap and hierarchical clustering of extracted image features. (B) Selected single features describing cellular changes upon increasing (OEG₂)₂-IP4 concentrations are depicted. Total cell count, dead cell count, single cell area and single cell solidity, a measure of cell compactness, are shown. (C) Selected single features describing changes in the CaP deposition and membrane pattern upon increasing (OEG₂)₂-IP4 concentrations are depicted. The total area of the binary calcein staining, colorimetric quantification of calcium content extracted from the monolayer, the maximum intensity of the calcein fluorescence, structural similarity index metric (SSIM) and the correlation of the CellMask channel image are shown. (D) Example brightfield, calcein, CellMask and EthD channel images and two zoomed-in regions of interests (ROI) of RPTEC treated with 13 μM (OEG₂)₂-IP4 are shown. Mean scaled values per individual experiment are plotted (N=3). Pos.ctrl presents treatment with 5/7 mM Ca/P, neg.ctrl presents medium without Ca/P spiking (=1/1 mM Ca/P present in normal cell medium). Mean of three individual experiments and SD is plotted as grey horizontal and vertical line respectively, one-way ANOVA with Dunnet's multiple comparison between each concentration to pos. ctrl was performed (* p<0.01).

FIG. 5 shows Ca/P-induced transcriptomic changes of renal epithelial cells include inflammatory pathways, ECM proteins, cell proliferation and tissue homeostasis processes and are prevented by (OEG₂)₂-IP4 in vitro. A) Heatmap and hierarchical clustering of relative gene expression levels determined by RNA sequencing. Top 2000 differentially expressed genes between Ca/P vs. medium control with p≤0.01 and log 2fold change≥0.5, and respective expression levels in the other treatment groups are plotted (red-relatively upregulated, blue-relatively downregulated). B) Overrepresentation analysis of upregulated gene transcripts in the Ca/P vs. medium group. C) Overrepresentation analysis of downregulated gene transcripts in the Ca/P vs. medium group. Weighted set coverage of gene ontology terms and their respective enrichment score for differentially expressed genes in the Ca/P vs. medium control group is plotted (all FDR≤0.05). D) Normalized count of gene transcripts (FPKM—fragments per kilobase of exon model per million reads mapped) involved in inflammatory responses, ECM composition, cell proliferation and tissue homeostasis of the different treatment groups is plotted (mean+SD, N=3).

FIG. 6 shows (OEG₂)₂-IP4 reduces high phosphate induced kidney damage in vivo. C57BL/6 male were placed on either regular diet containing 0.35% inorganic phosphate (NP) or high phosphate diet containing 2.0% inorganic phosphate (HP). These mice were subcutaneously injected with either (OEG₂)₂-IP4 (100 mg/kg) or vehicle (distilled water) three times a week and then sacrificed at 20 weeks of age to harvest their blood and kidneys. Relative mRNA levels of (A) Spp1, (B) IL36a, (C) Ngal, (D) MMP3 and (E) Col1a1 in kidney tissue homogenate are shown (mean±SD, N=7, except HP vehicle group N=6, two-way ANOVA with Sidak's multiple comparison between (OEG₂)₂-IP4 vs. vehicle control within the respective diet group, ns—not significant, * p<0.05). (F) Collagen volume fraction following Picro-Sirius red staining of the kidneys (mean±SD, N=6, t-test between (OEG₂)₂-IP4 vs. vehicle, ** p<0.01).

FIG. 7 shows inhibition of CaP precipitation with OEG₂-IP5, OEG₁₁-IP5, (OEG₂)₂-IP4, (OEG₁₁)₂-IP4 and OEG₃-(IP5)₂. Effects of (A) OEG₂-IP5, (B) OEG₁₁-IP5, (C) (OEG₂)₂-IP4, (D) (OEG₁₁)₂-IP4 and (E) OEG₃-(IP5)₂. on CaP precipitation in RTF spiked with 9 mM disodium phosphate and 8 mM calcium chloride were assessed by light microscopy followed by automated image analysis at t=4 h. Quantification of the total area covered with CaP precipitates/total area field of view (N=3, mean+SD, normalized to the control).

FIG. 8 shows inhibition of CaP aggregation with OEG₂-IP5, OEG₁₁-IP5, (OEG₂)₂-IP4, (OEG₁₁)₂-IP4 and OEG₃-(IP5)₂. Effects of (A) OEG₂-IP5, (B) OEG₁₁-IP5, (C) (OEG₂)₂-IP4, (D) (OEG₁₁)₂-IP4 and (E) OEG₃-(IP5)₂. on CaP precipitation in RTF spiked with 9 mM disodium phosphate and 8 mM calcium chloride were assessed by light microscopy followed by automated image analysis at t=4 h. Quantification of the mean size of aggregates/field of view normalized to the ctrl (N=3, mean+SD, normalized to the control).

FIG. 9 shows in vitro reduction of CaP adhesion and prevention of cell injury by IP6 analogues. RPTEC/TERT 1 monolayers at confluence were treated with medium spiked with 7 mM disodium phosphate, 5 mM calcium chloride and compound and were incubated for 24 h. The amount of CaP deposits and the extent of cell injury were detected by calcein and EthD staining, respectively. Fluorescence images were quantified using Matlab. Quantification of the area covered with CaP deposition on RPTEC monolayers with (A) OEG₂-IP5, (B) OEG₁₁-IP5, (C) (OEG₂)₂-IP4, and (D) OEG₃-(IP5)₂ treatment. (N=3, mean+SD, one-way ANOVA with Dunnett's multiple comparison, * p<0.05, ** p<0.01, *** p<0.001).

FIG. 10 shows example images of RPTEC treated with 5/7 mM Ca/P and increasing concentrations of (OEG₂)₂-IP4. Brightfield, calcein, CellMask™ and EthD channel images and two zoomed-in regions of interests (ROI) are shown (column 1-4). Cell segmentation is shown overlayed with the Hoechst channel image (blue) (column 4). Pos.ctrl presents treatment with 5/7 mM Ca/P, neg.ctrl presents medium without Ca/P spiking (=1/1 mM Ca/P present in normal cell medium).

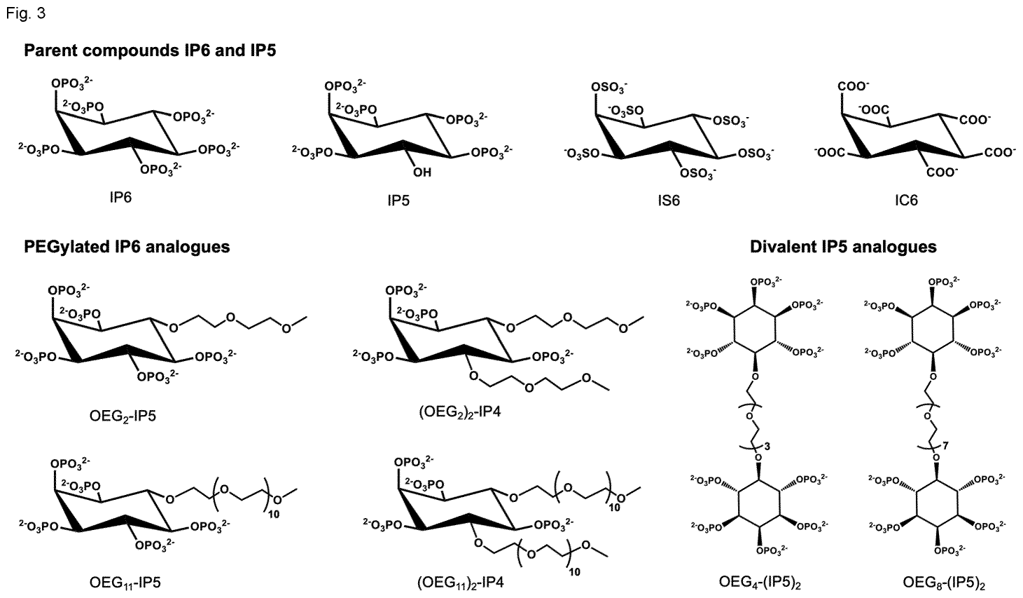

Tab. 1 shows an overview of the efficacy of screened compounds to inhibit CaP precipitation in RTF. Overview table showing the minimal concentration of compound (FIG. 3 for the chemical structures) required to achieve complete inhibition of CaP precipitation (total area covered with crystals <10% of total area control); and minimal concentration required to prevent CaP crystal aggregation in the CaP screening assay (CaP aggregate size <50% of aggregate size control) (N=3).

Tab. 2 shows serum and urinary phosphate and calcium levels measured in the different treatment groups of mice (mean±SD).

### EXAMPLES

Material and Methods

Materials

IP6 analogues were custom synthesized by Chimete Srl (Tortona, Italy). Mass and ¹H-NMR spectra were taken by the provider to confirm the structure and the compounds were used as provided. Phytic acid dodecasodium salt was purchased from Biosynth AG (Thal, Switzerland). IP5 and IS6 hexapotassium salt were purchased from Santa Cruz Biotechnology (Dallas, Texas, United States). IC6 was purchased from Fluorochem (Hadfield, United Kingdom). Calcium Colorimetric Assay kit (MAK022), Bis-Tris, sodium oxalate (NaOx), EthD, Hoechst 33342, magnesium chloride hexahydrate, sodium phosphate dibasic, and calcein were purchased from Sigma-Aldrich (St.Louis, MO, USA). Sodium chloride, sodium sulphate anhydrous and calcium chloride (CaCl₂) dihydrate were obtained from Merck (Kenilworth, NJ, USA). Calcium oxalate (CaOx) monohydrate was purchased from abcr (Karlsruhe, Germany). 8-well glass bottom slides (80 827) were purchased from ibidi (Martinsried, Germany). CellMask Deep Red Plasma Membrane Stain, standard cell culture plates and reagents were purchased from Thermo Fisher Scientific (Rochester, NY, USA) and TPP (Trasadingen, Switzerland). RPTEC/TERT1 cells, ProxUp basal medium, and supplements were obtained from Evercyte (Vienna, Austria). RNeasy kit was purchased from Quiagen (Hilden, Germany) and TrueSeq RNA kit from Illumina (San Diego, CA, USA). RNAiso Plus was obtained from TaKaRa (Kusatsu, Japan). ReverTra Ace qPCR RT Master Mix with gDNA Remover and SYBR Green PCR Master mix were purchased from Toyobo (Osaka, Japan).

In-Solution Screening

An artificial renal tubular fluid (RTF) with a final composition of 0.05 mM oxalate, 0.005 mM sulphate, 408 mM sodium, 424 mM chloride, 0.26 mM potassium, 4 mM magnesium and 0.2 mM citrate in double distilled water was prepared according to a literature report (Fasano, J. M. et al., Kidney Int. 59, 169-178. DOI: 10.1046/j.1523-1755.2001.00477.x, 2001). The solution was filtered with a 0.45-μm syringe filter. Twenty mM Bis-Tris buffer was added to the reported protocol to ensure pH stability throughout experiments and pH was set to 7.2. The artificial RTF was stored at room temperature for up to four months. Stock solutions of 0.25 M phosphate and 1 M calcium were prepared in double distilled water and were stored separately at −20° C.

Twenty-x final concentration phosphate, 20× final concentration calcium and 10× final concentration compound dilutions were prepared in RTF. Assay mixture consisting of 80% RTF, 10% compound dilution, 5% phosphate dilution and 5% calcium dilution was prepared in Eppendorf tubes as follows. RTF (320 μL) was mixed with 20 μL phosphate dilution (final concentration of 9 mM), 40 μL compound dilution and 20 μL calcium dilution (final concentration of 8 mM). The assay mixture was vortexed after adding each component and 380 μL of the mixture were immediately added to 8-well glass bottom slides and incubated for 4 hat RT. CaP precipitation was assessed using a Leica DM 6000B microscope (Leica Microsystems, Wetzlar, Germany) in brightfield mode. For quantification 3 wells/condition with 3-4 images/well were imaged with a 40× objective. The total area covered with CaP deposits in percentage of the field of view and the mean size of CaP aggregates were determined.

Cell Culture

RPTEC/TERT 1 human proximal tubule cells (RPTEC) were cultured in T75 tissue culture flasks using ProxUp basal medium mixed with ProxUp supplements at 37° C. and 5% CO2 according to manufacturer's recommendations. Cells were used up to passage 30 and regularly tested for Mycoplasma infections. For experiments, RPTEC were cultured in 24-well plates at a seeding density of 150′000 cells/cm². Cell viability assessment and cell counting before seeding was performed using an automated cell counter (BioRad TC 20, Hercules, CA, USA).

Imaging Assay

At t=48 h after seeding, RPTEC were treated with ProxUp basal medium spiked with first, various concentrations of phosphate (final concentration between 1 and 7 mM) and second, calcium (final concentration between 1 and 7 mM), which was directly added to each well.

For the comparison of selected IP6 analogues, ProxUp basal medium was prepared with the selected inhibitor and added to each well. Then, CaP precipitation was induced by direct addition of first, phosphate and second, calcium. Final concentrations of 7 mM phosphate and 5 mM calcium were used. Cells were incubated for 24 h at 37° C. and 5% CO₂. Spiked medium was removed, and cells were washed twice with PBS before staining.

For the assessment of CaP-induced CaOx crystallization, after 24 h of incubation, Ca/P spiked medium was removed, and cells were washed twice with PBS. Subsequently, medium containing 1.2 mM oxalate and compound was added to each well. After 4 h of incubation at 37° C. and 5% CO₂ treatment was removed, and cells washed once with PBS before staining.

For staining, ProxUp basal medium was mixed with calcein (500 nM final concentration), EthD (6 μM final concentration), CellMask stain (5 μg/μL) and Hoechst 33342. Staining mixture was added to RPTEC and cells were incubated for 30 min in the dark at 37° C. and 5% CO₂. Staining solution was removed, cells were washed with PBS once and ProxUp basal medium was added. Cells were immediately imaged after staining. Images were obtained by epifluorescence microscopy at 37° C., using a Leica CTR6000 microscope. For quantification 3 wells/condition were prepared and 3 images/well were taken. For preliminary adhesion experiments images were taken with a 10× objective, for the imaging assay 20× objective images were taken.

Calcium Colorimetric Quantification

For colorimetric quantification of the calcium content of cell monolayers after Ca/P treatment a previously reported protocol was used. In brief, after 24 h of Ca/P incubation cell monolayers were stained and imaged as described above. Then, cells were washed 1× with PBS before overnight incubation with 250 μL of 0.1 M HCl at 4° C. to decalcify cell monolayers. HCl solution was collected, centrifuged at 10,000×g, 4° C. for 4 min and calcium content determined using the Calcium Colorimetric Assay kit (MAK022).

Cell Image Analysis

Multichannel images were saved as individual channel images in 8-bit tiff format. For analysis Hoechst channel images were thresholded using the triangle threshold. Touching nuclei of the binary image were further segmented using the watershed algorithm with the distance transform of the binary image as input and local maxima thereof as seeds.

For EthD channel images, high level of background noise was observed, potentially as a result of fluorescence of the CellMask stain bleeding through. Thus, it was found that using the 99 percentile of all intensity values per image as a threshold resulted in a good detection of foreground pixels. A fixed minimum threshold was set at 60. Watershed segmentation similar to the Hoechst channel was performed to segment touching objects. For the calcein channel images adaptive thresholding by triangle thresholding achieved good detection of CaP deposits, which was validated by visual comparison to brightfield channel images. For compound experiments, an additional upper threshold was set to the minimum threshold of the pos.ctrl images for the respective experiment. Thereby an overly insensitive threshold in images with a high amount of CaP deposition was circumvented. The sum of foreground pixels of the binary image was used to calculate the total area of CaP deposits. The skimage label algorithm was used to label touching foreground regions.

To segment images to single cells the CellMask channel image was first binarized by adaptive thresholding, using a blocksize of 35, followed by median filtering. Binary erosion was performed to shrink the outlines. A version of watershed algorithm was used to segment the whole image into single cells. To this end, the distance transform of the binary erosion image was used as input image and seeds were set to the local maxima of the Hoechst channel image. While this protocol allowed for an approximation of single cell morphology, further improvements of both analytical and experimental staining procedure would be necessary to achieve more accurate results.

Additionally, texture features of calcein and CellMask channel images were extracted. Grey level co-occurrence matrices were calculated using one set offset of 5 pixel and an angle of 90°. Here, the use of larger offsets could improve the detection of changes in large-scale features, e.g. CaP clustering sites. Texture properties of the matrices extracted were contrast, dissimilarity, energy, ASM, homogeneity and correlation. Overlap between calcein and CellMask channel images was measured by computing the structural similarity index matrix (SSIM).

Features extracted included both single cell or single calcein patch features, as well as whole image features. Whole image features included a total cell count based on the Hoechst image, a total dead cell count based on the ethd channel image, SSIM, texture features of both calcein and CellMask channel images and maximum, minimum, mean and standard deviation, total area and cluster count of calcein intensities. Single cell, single nuclei and single calcein patch features included shape and, only for calcein and cell, intensity properties and were summarized to median values per image. Properties included median area, extent, eccentricity, perimeter, solidity, major and minor axis length, as well as maximum, minimum and mean intensity per cell or CaP patch. Features were scaled ranging from 0 to 1 using the MinMaxScaler from the sklearn preprocessing package. The mean value of each image feature over 9 images (3 wells*3 images/well for each condition) per condition was calculated for each experiment and three independent experiments used for the final analysis. Analysis was performed in Python 3 using the numpy, pandas, skimage, sklearn and seaborn packages.

RNA sequencing Cells were cultured as described in the imaging assay and treated with a medium control (ProxUp basal medium containing 1/1 mM Ca/P), 5/7 mM Ca/P, 5/7 mM Ca/P in medium containing 50 μM (OEG₂)₂-IP4 or 50 μM (OEG₂)₂-IP4 in medium for 24 h. Total RNA was extracted using the RNeasy kit (Quiagen) according to the manufacturer's instructions. Three wells per sample group were prepared and total RNA extracted of those 3 wells pooled. mRNA was purified and RNAseq library was prepared using the TrueSeq RNA kit (Illumina). Sequencing was performed on a Novaseq 6000 (Illumina). Reads were aligned to the human reference genome GRCh38.p10 using the STAR tool (https://github.com/alexdobin/STAR) and transcripts quantified using the Kallisto program (42). Ensembl release 91 was used for the gene model definitions. For the heatmap and hierarchial clustering of significantly different genes (p≤0.01, log 2fold change≥0.5) the log₂-fold changes in comparison to the mean of all samples was calculated and log 2fold changes >4 were set to 4. The heatmap was plotted using R software. Overrepresentation analysis was performed on Webgestalt.org (v2019) (43), using differentially expressed genes with p≤0.01, log 2fold change≥0.5. Differential expressed genes were compared to the gene ontology—biological process functional database and as a reference set the human genome—protein coding was used. The weighted set cover of the top 30 enriched categories was plotted. For comparison of expression levels of the selected genes, the fragments per kilobase of exon model per million reads mapped (FPKM) were used. Three independent experiments were performed. RNA sequencing raw data is available on the EMBL Nucleotide Sequence Database (ENA) under the accession number PRJEB38397.

Animal Studies

C57BL/6 male mice (12 weeks of age) were placed on either regular diet containing 0.35% inorganic phosphate or high phosphate diet containing 2.0% inorganic phosphate. These mice were subcutaneously injected with either (OEG₂)₂-IP4 (100 mg/kg) or vehicle (distilled water) three times a week and then sacrificed at 20 weeks of age to harvest their blood and kidneys. Some mice were transferred individually to metabolic cages to collect urine for 3 days before sacrifice. Serum FGF23 levels were measured using intact FGF23 ELISA (Kinos) according to the manufacturers' protocols. Serum and urine levels of phosphate were measured using Fuji Dri-Chem slides and the analyzer (Dri-Chem NX500V, Fuji, Tokyo, Japan). Frozen mouse kidneys were homogenized with RNAiso Plus (Takara, Osaka, Japan). The lysates were extracted with chloroform. RNA in the aqueous phase was precipitated with isopropanol, washed with 75% ethanol, and dissolved in RNase-free water. Reverse transcription of RNA (0.4 μg) was carried out using ReverTra Ace qPCR RT Master Mix with gDNA Remover (Toyoba, FSQ-301, Osaka, Japan) according to the manufacturer's protocol. Quantitative RT-PCR reactions were performed using 20 ng of cDNA incubated with 410 nM of each primer and 6 μL of SYBR Green PCR Master mix (Toyoba, Osaka, Japan THUNDERBIRD SYBR qPCR Mix QPS-201) in a total volume of 12 μl. The PCR reaction (95° C. for 1 minute followed by 45 cycles of 95° C. for 10 s, 60° C. for 40 s) was carried out on a Roche LC480 system (Basel, Switzerland). Relative mRNA levels were calculated by the comparative threshold cycle method using cyclophilin as an internal control. Primer sequences can be found in STable 6. The kidneys not used for RNA extraction were fixed in 10% formalin, processed to make standard paraffin sections, and stained with Picro-Sirius Red to detect collagen as red fibers. The collagen volume fraction (the ratio of the Sirius Red-positive area to the total area) was quantified using an image analysis software (IMAGE PRO 9.32, Medica Cybernetics, Rockville, MD, USA) as previously described (Hirano, Y. Kurosu et al., FEBS Open Bio. 10, 894-903. DOI:10.1002/2211-5463.1284, 2020). The cortex and the cortico-medullary junction were evaluated separately. All animal experiments were approved by the institutional animal care and use committee from Jichi Medical University.

Data Analysis

All images were analysed and graphs prepared using Python 3, except pre-screening in solution experiments and preliminary cell adhesion data were analysed using Matlab and plotted using GraphPad Prism (GraphPad, La Jolla, CA, USA). RNA sequencing data was analysed as described in the above section and graphs prepared using R software and GraphPad Prism. Animal data were analysed in GraphPad Prism.

Results

### Example 1: Image-Based Profiling of Calcification Processes

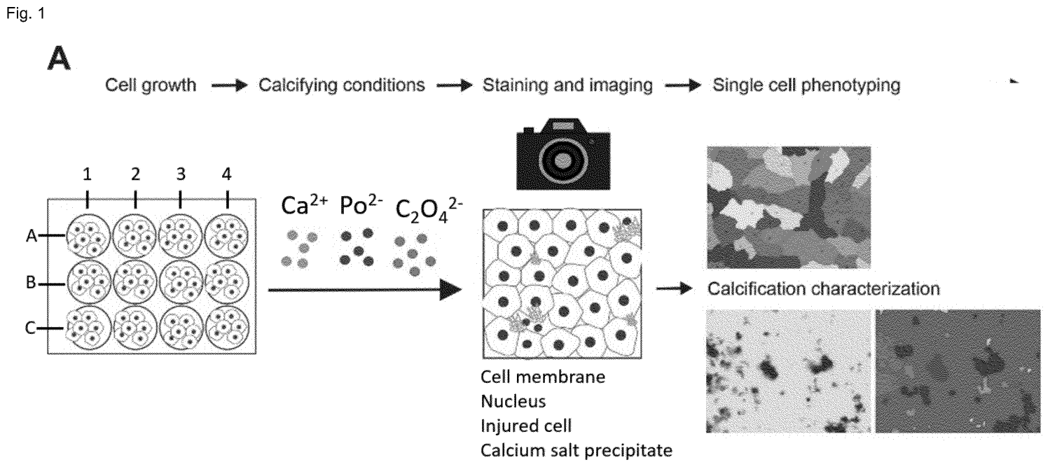

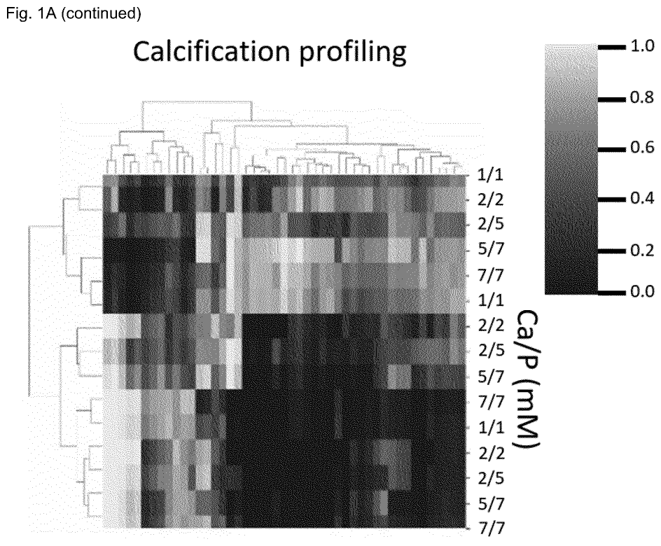

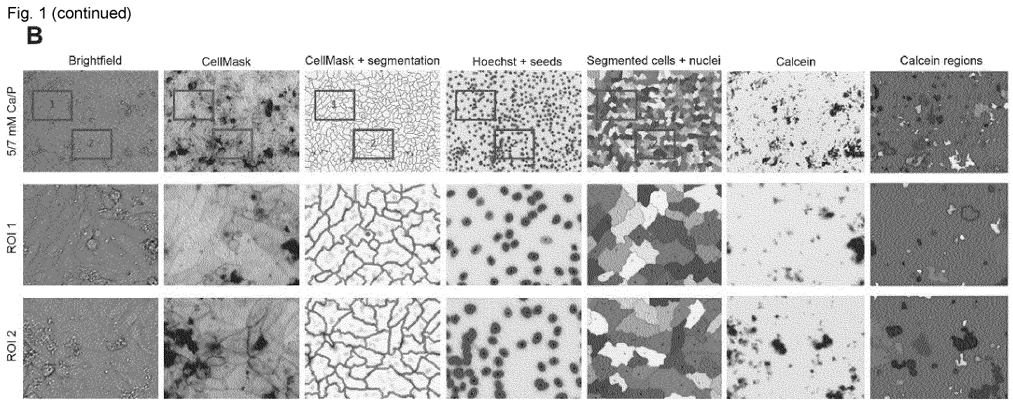

To allow rapid profiling of molecules for their effects on diverse kidney calcification processes, the inventors developed a cell-based assay that allowed the monitoring of CaP deposition, as well as cellular changes associated with it. Therefore, the inventors utilized monolayers of renal proximal tubular cells (RPTEC) stained with various dyes to quantify calcification and cell morphology changes (FIG. 1). Cells grown in monolayer were exposed to varying ionic conditions found within the renal tubules, e.g. increased calcium and/or phosphate, to trigger the crystallization of CaP and cellular attachment (FIG. 1A). CaP deposits were detected via calcein staining (FIG. 1B). Calcein has been previously suggested as a calcium staining technique of fixed or unfixed cell samples. The fluorescent dye binds to calcium and is imaged using fluorescent microscopy. Further, the inventors tested the induction of CaP-induced CaOx crystallization, which is characteristic for idiopathic kidney stone formation. It was observed that by first inducing CaP deposition, followed by addition of high oxalate, CaOx crystallization can be found on CaP deposits. CaOx crystals displayed a strong contrast and a typical twinned structure.

Cellular changes were visualized by staining for the membrane with a CellMask dye. Hoechst was used as a nuclear dye, to aid in single cell segmentation, and ethidium-homodimer 1 (EthD) facilitated the staining of cells with a damaged plasma membrane (FIG. 1B). Further, texture features of both the CellMask and calcein channel were extracted, which were indicative of CaP deposition patterns (i.e., large and high intensity CaP clusters vs. more diffuse and equally spread CaP across the cell monolayer) (FIG. 1B).

### Example 2: CaP-Induced Changes on Renal Epithelial Cells

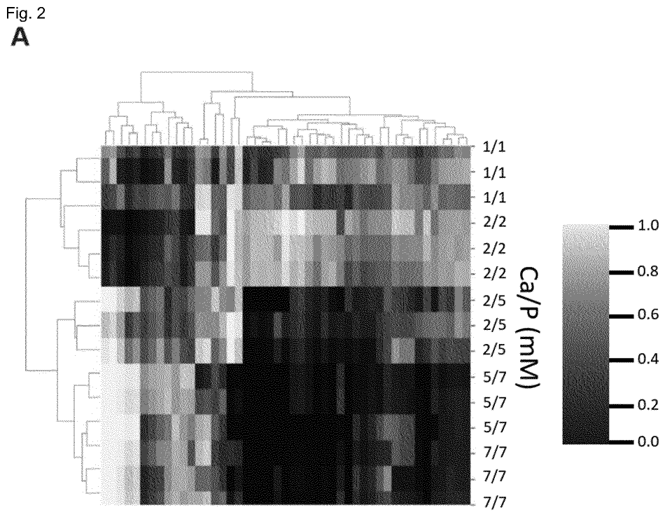

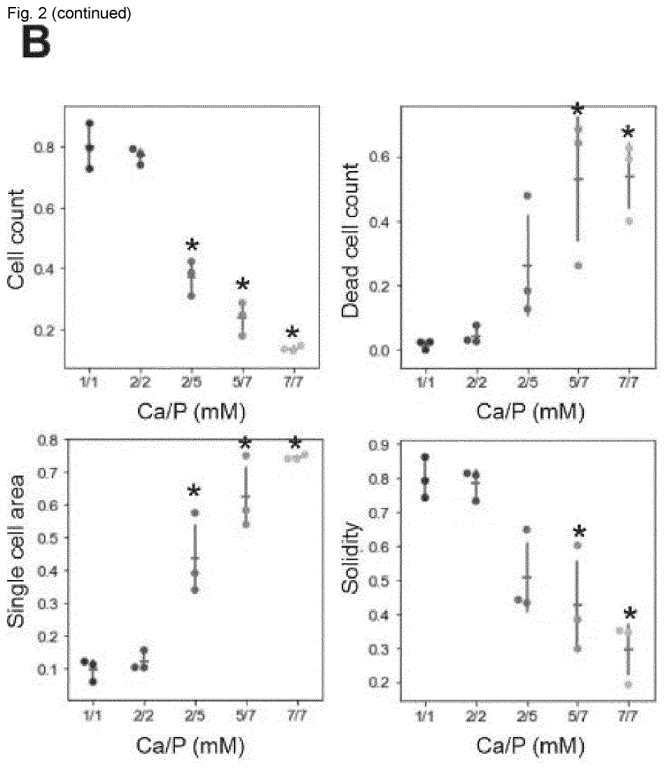

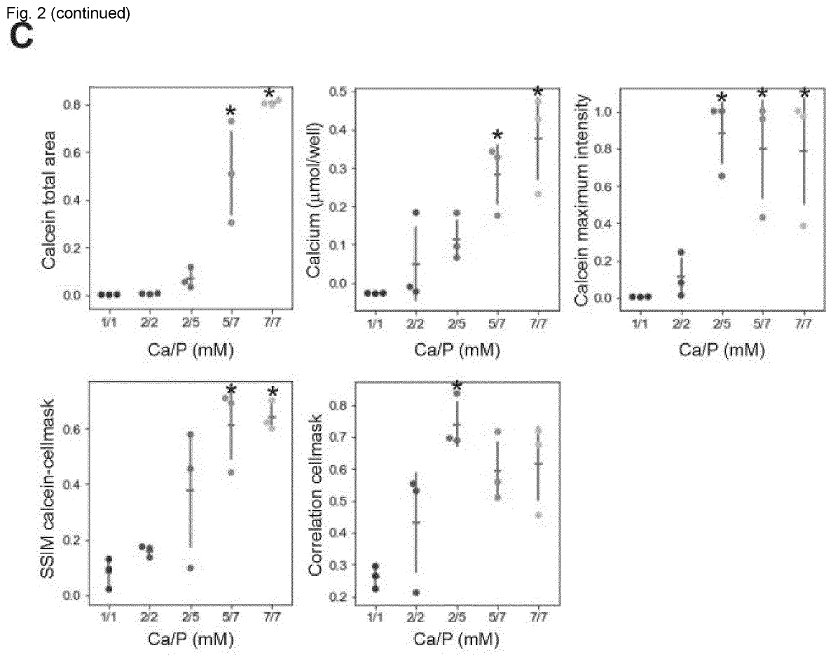

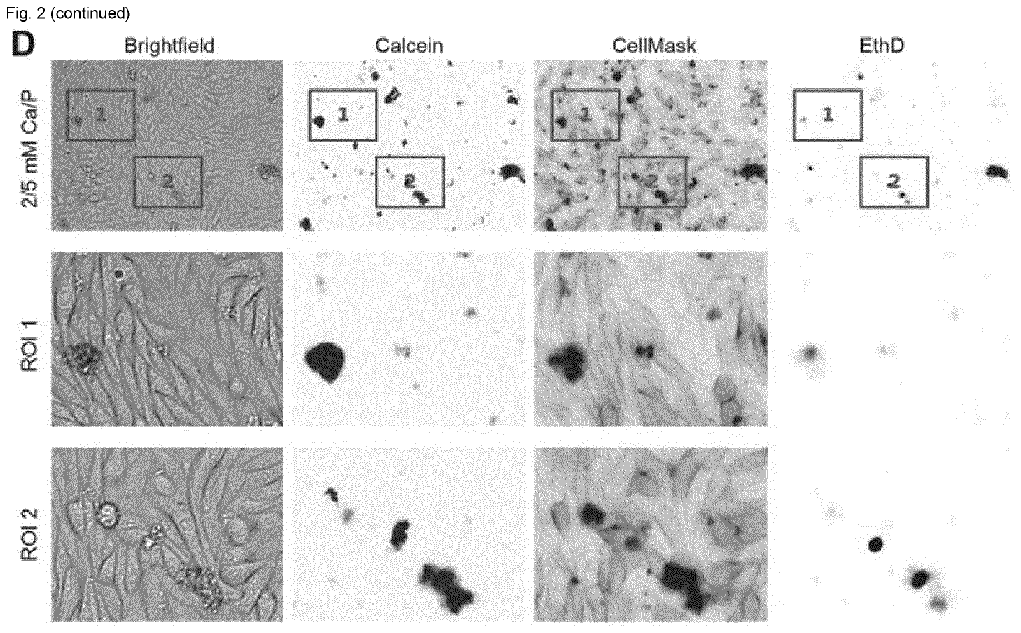

The inventors first investigated the effects of increasing calcium and phosphate concentrations on cellular CaP deposition and the ensuing cellular changes. Features extracted included single cell shape and fluorescence intensity parameters, texture features of both the CellMask and calcein images, and CaP deposit shape and intensity features. Hierarchical clustering of experimental conditions and features showed a clear dose-dependent trend (FIG. 2A). Low concentrations of calcium and phosphate, such as those of non-spiked cell media (1/1 mM Ca/P) and low spiked one (2/2 mM Ca/P) did not induce cellular changes, while higher levels resulted in intermittent (2/5 mM) and drastic changes (5/7 and 7/7 mM) (FIG. 2B). Cellular changes associated with Ca/P levels became more evident when looking at single parameters. Increasing Ca/P led to loss of tight packing of cells, characteristic for the renal epithelium, which is associated with an increase in single cell area and a decrease in single cell solidity, a measure for compactness. Hence, these data indicated the loss of the typical round shape of the epithelial cells towards a more irregular shape (FIG. 2B). These effects were accompanied by a decrease in the total number of cells/field of view. Additionally, an increase in cells with a damaged plasma membrane was detected with increasing Ca/P levels (FIG. 2B). These results are in agreement with literature reports, which suggest a loss of epithelial phenotype and cell injury upon stimulation with CaP or CaOx.

Levels of CaP deposition were measured first by, adaptive thresholding of the calcein image, and second, quantification of the total area covered per field of view. Using this approach, the inventors found an increased coverage of the cell monolayer with CaP upon higher Ca/P concentrations (FIG. 2C). These results were qualitatively validated by comparison with brightfield images, where CaP is visible as a feature with a granular texture, or, in the case of large deposits, as dark spots (FIG. 2D). Calcein quantification of CaP deposition was confirmed by employing a colorimetric quantification of calcium, which was extracted from the cell monolayer by acid treatment (FIG. 2C) (Schantl, A. E. et al., Nat. Commun. 11, 721. DOI: 10.1038/s41467-019-14091-4, 2020). Interestingly, while treatment with 2/5 mM Ca/P did not lead to a great enhancement of area of CaP depositions, the deposits were of high calcein intensity (FIG. 2C, D). Thus, the inventors hypothesized that rather than equally distributing across the monolayer, at lower concentrations, CaP tends to accumulate at sites of enhanced CaP affinity, such as dedifferentiating or injured cells with increased surface expression of glycoproteins with calcium binding properties. These findings were supported by a high structural similarity index metric (SSIM) of the calcein and CellMask channel image, indicating the overlap of areas of CaP deposition with membrane staining, upon intermediate and high Ca/P stimulation (FIG. 2C). The overlapping areas showed a high intensity of membrane staining, which could indicate clumps of cell debris of injured and detaching cells. Additionally, the correlation texture feature of the CellMask channel image, reflecting consistency of an image, showed an increase between 1/1 to 2/5 mM Ca/P spiking, before a drop occurred again at ≥5/7 mM Ca/P (FIG. 2C). The highest value observed for the intermediate Ca/P concentration might be due to the loss of cell outlines when Ca/P is present but no large CaP cluster sites were formed. At high concentrations, CaP sites again caused a decrease in the correlation feature due to CaP-membrane clusters giving high staining intensities. For the further testing of inhibitors, concentrations of 5/7 mM Ca/P were used, which are within the physiological range in the loop of Henle, the suggested main site of CaP crystallization.

### Example 3: Pre-Screening of IP6 Analogues as Renal CaP Inhibitors in Solution

In a first step, the influence of selected IP6 analogues (FIG. 3) on CaP precipitation and growth was assessed in vitro using artificial renal tubular fluid (RTF) (Table 1). In a previous study, the inventors investigated CaP protein particle formation in serum. However, an important difference to cardiovascular calcification lies in the extremely low protein content of the renal tubular fluid vs. the high abundance of proteins in blood, which can stabilize amorphous particles. Two cut-offs were chosen for assessing efficacy. First, complete inhibition was defined as a >90% reduction of detected CaP precipitates compared to the control sample without compound. Second, inhibition of CaP aggregation was defined as a >50% reduction of the mean size of aggregates when compared to the control.

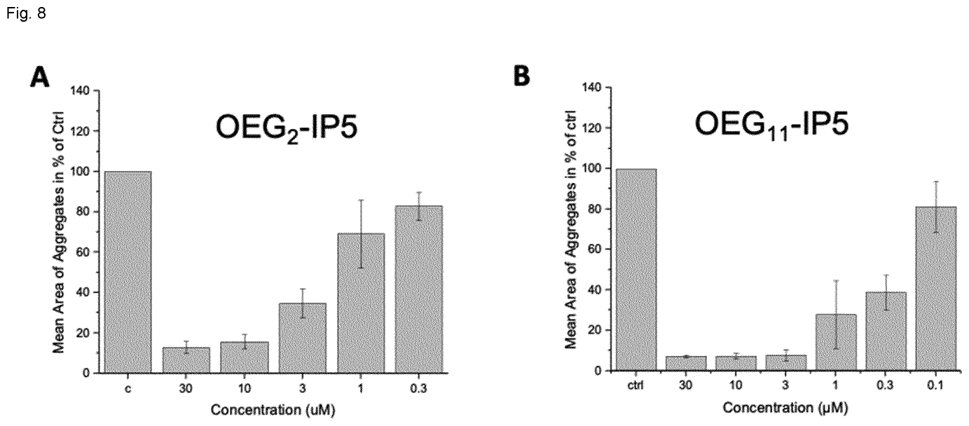

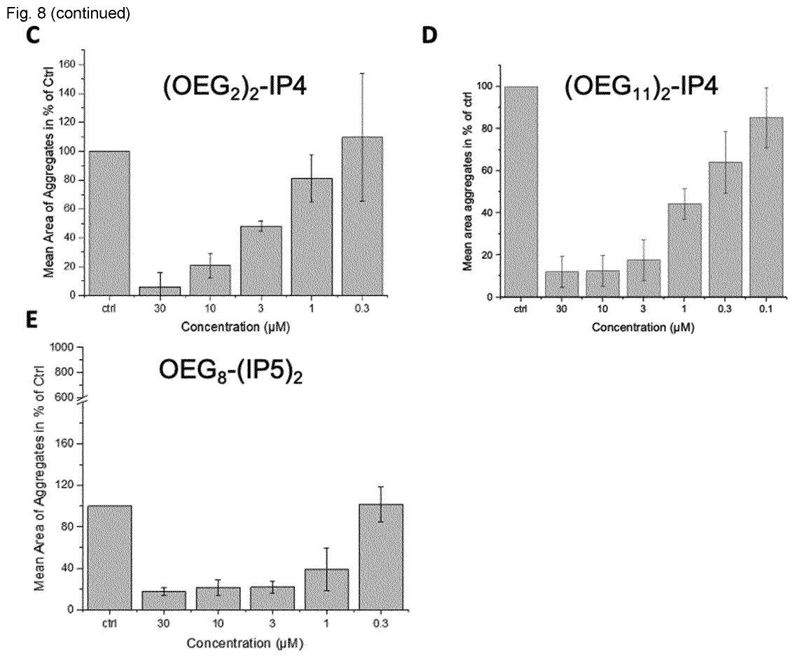

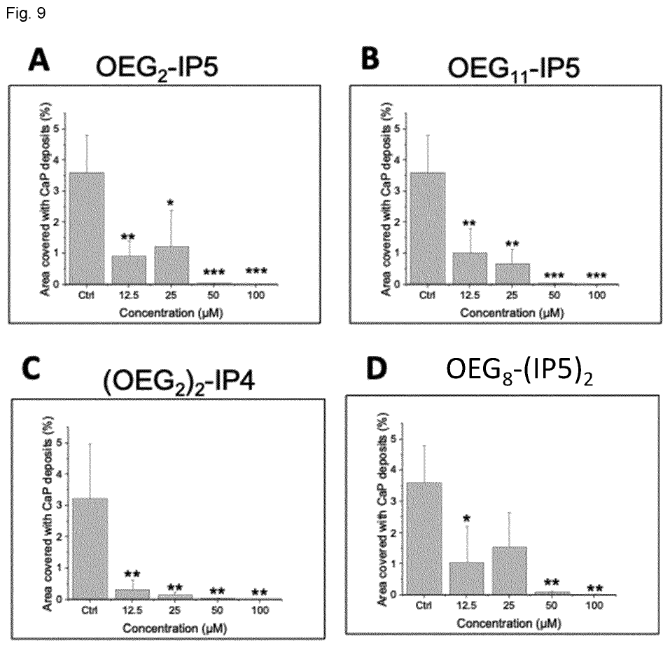

Interestingly, IP6, and to a lesser extent myo-inositol pentakisphosphate (IP5), promoted CaP precipitation at 10 and 30 μM, respectively. CaP precipitation was likely accelerated by IP6-calcium aggregates forming, as the inventors confirmed in media without phosphate. Replacing phosphate groups with oligoethylene glycol (OEG) chains, as in the case of OEG₂-IP5, led to inhibition of CaP precipitation and aggregation at 30 and 1 μM, respectively (Table 1, FIGS. 8A and 9A). Comparing OEG₂-IP5 and OEG₁₁-IP5 revealed that increasing the length of the OEG chain from 2 to 11 repeating units improved the molecule's inhibitory properties, reducing its aggregation inhibitory concentration from 1 μM to 300 nM (Table 1, FIGS. 8B and 9B). However, further phosphate substitution with OEG ((OEG₂)₂-IP4 vs. OEG₂-IP5) did not further increase the inhibitory activity (Table 1, FIGS. 8C and 9C). The substitution of phosphate groups with less charged sulphate and carboxyl groups, e.g myo-inositol hexasulfate (IS6) and cyclohexane hexacarboxylic acid (IC6), led to a loss of the promoting effect. These compounds only exhibited weak inhibitory properties on CaP crystallization (Table 1).

Divalent IP5 molecules, which in a previous study have been identified as potent inhibitors of renal CaOx crystallization, revealed another interesting trend. The effect on crystallization depended on the length of the linker between the IP5 moieties. OEG₄-(IP5)₂, with 4 EG units in the linker, promoted CaP precipitation, while OEG₈-(IP5)₂ having 8 EG repeating units, had an inhibitory effect. Complete inhibition was observed at 30 μM and 50% aggregation inhibition obtained at 1 μM (Table 1, FIG. 8E, 9E). Together, these findings suggest that CaP inhibition by IP6 analogues in a medium completely devoid of protein is highly dependent on the molecules' charge and stabilizing properties.

### Example 4: OEG2)₂-IP4 Prevents CaP Deposition and Cellular Changes In Vitro

In the next step, the inventors compared OEG₂-IP5, OEG₁₁-IP5, (OEG₂)₂-IP4 and OEG₈-(IP5)₂ on their efficacy to prevent CaP adhesion (FIG. 9). Cell medium was spiked with compound before the addition of phosphate and calcium. Herein, efficacy was in a similar range for all compounds, achieving complete inhibition of CaP adhesion at 50 μM. Interestingly, (OEG₂)₂-IP4 performed best, drastically reducing CaP adhesion down to 12.5 μM, which together with its reported favorable pharmacokinetic profile (Schantl, A. E. et al., Nat. Commun. 11, 721. DOI: 10.1038/s41467-019-14091-4, 2020), led the inventors to further characterize this compound.

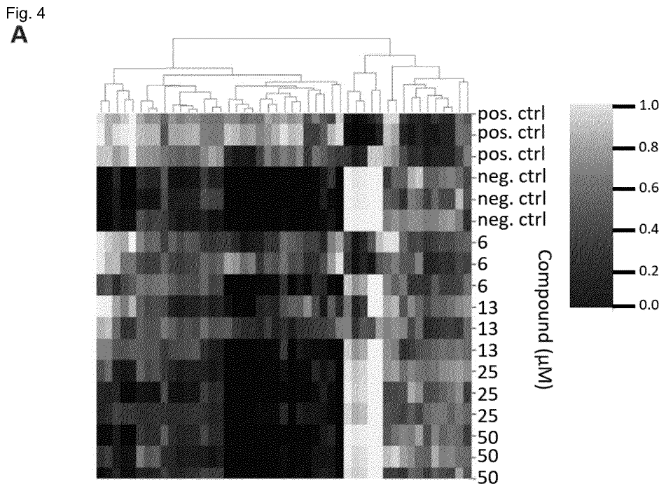

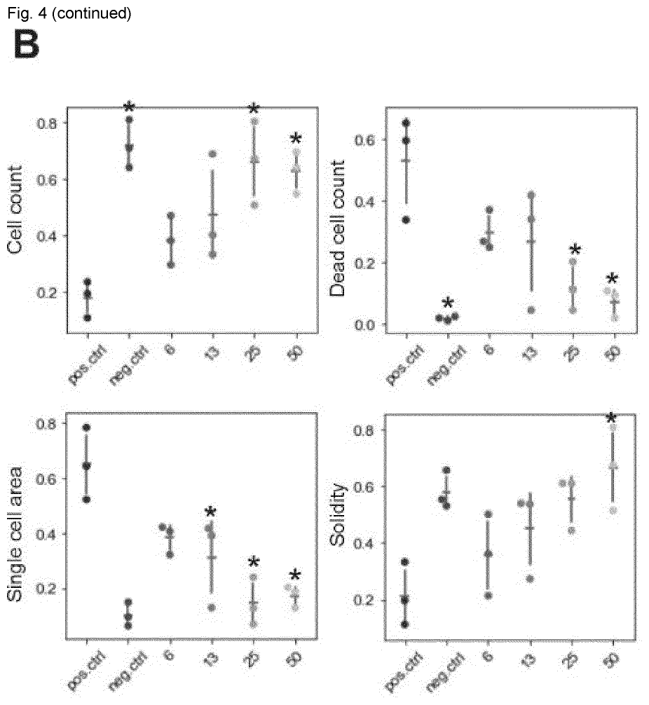

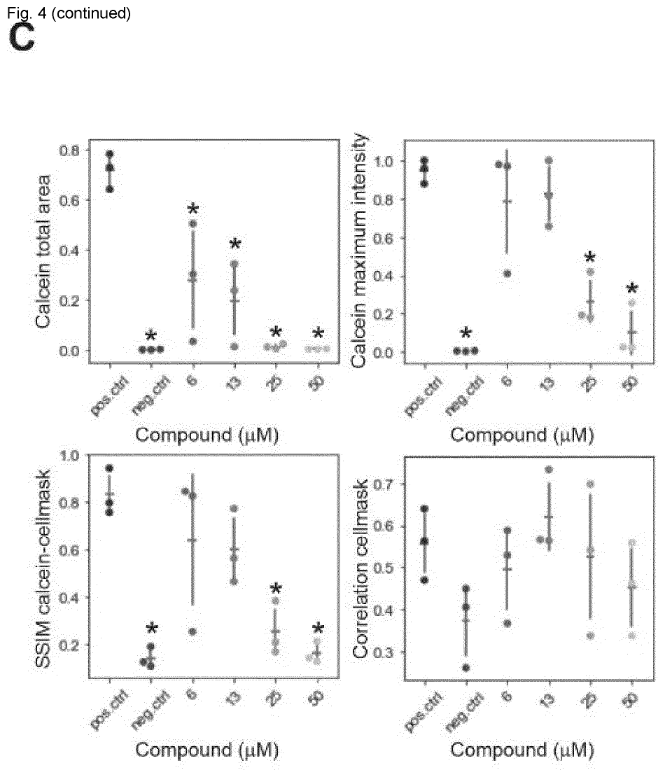

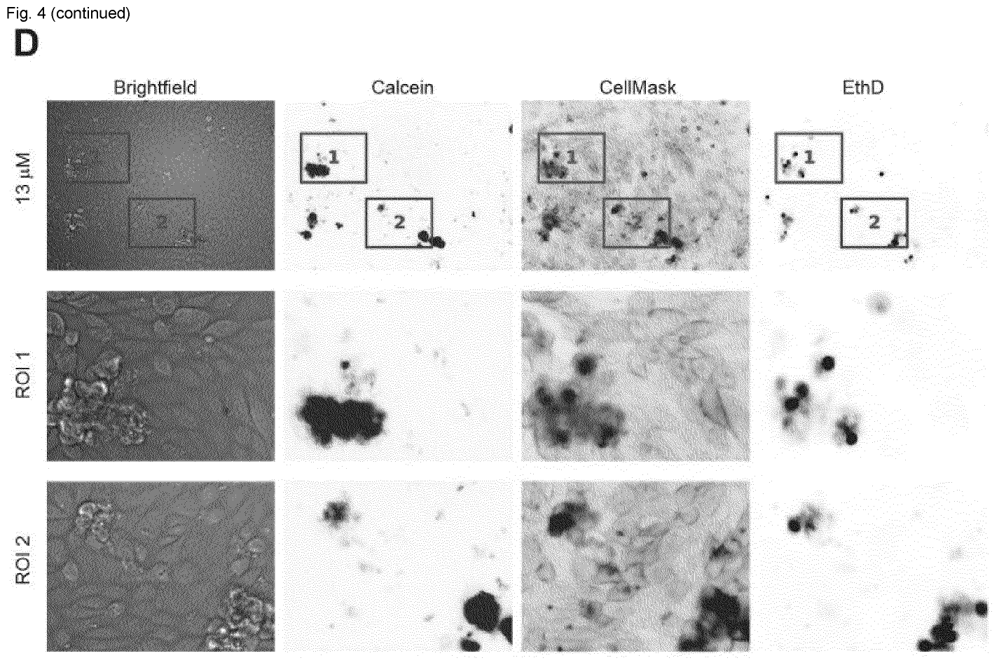

Treatment of cells with 6-50 μM of (OEG₂)₂-IP4 resulted in a dose-dependent reversal of the image feature profile from the positive control (ctrl) (+5/7 mM Ca/P) towards the negative ctrl (+1/1 mM Ca/P) (FIG. 4A). (OEG₂)₂-IP4 dose-dependently reduced the number of dead cells and single cell area compared to the positive ctrl, and increased single cell solidity and total cell count/field of view (FIG. 4B). Hence, these data suggest a reversal towards the negative ctrl phenotype with (OEG₂)₂-IP4.

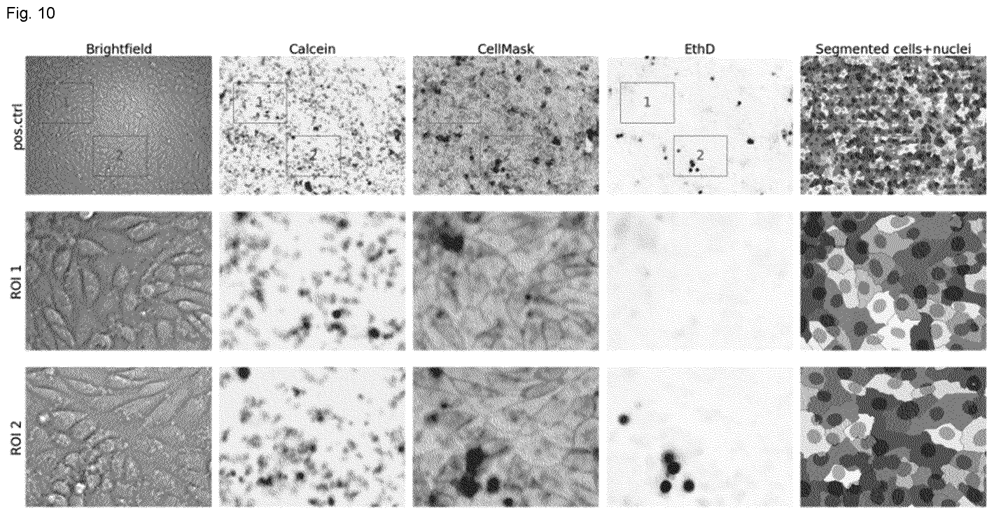

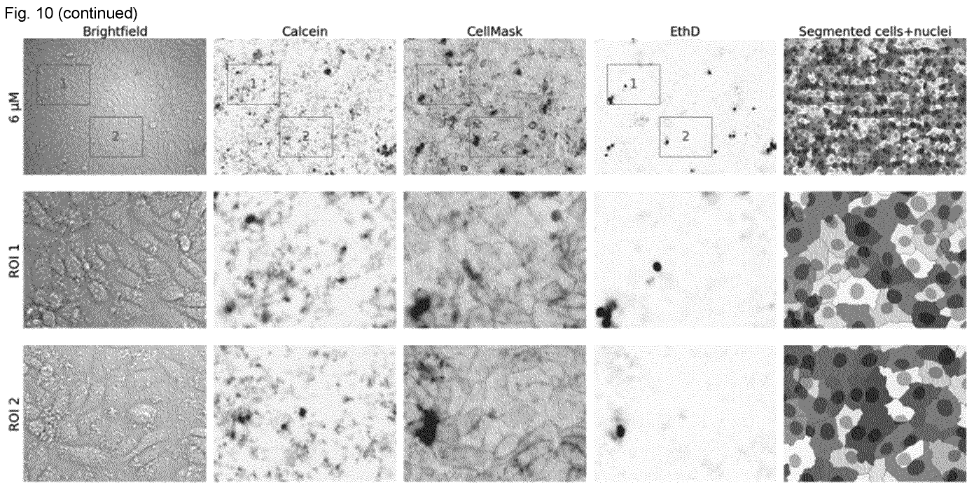

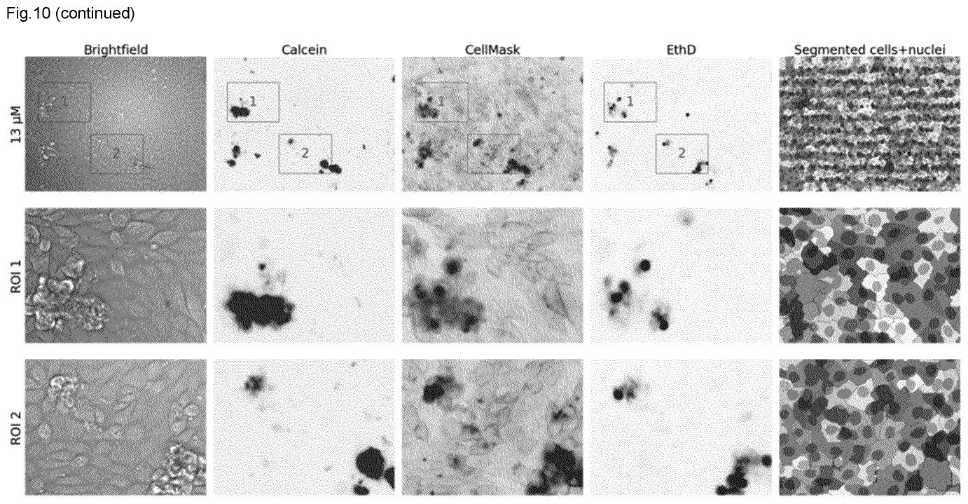

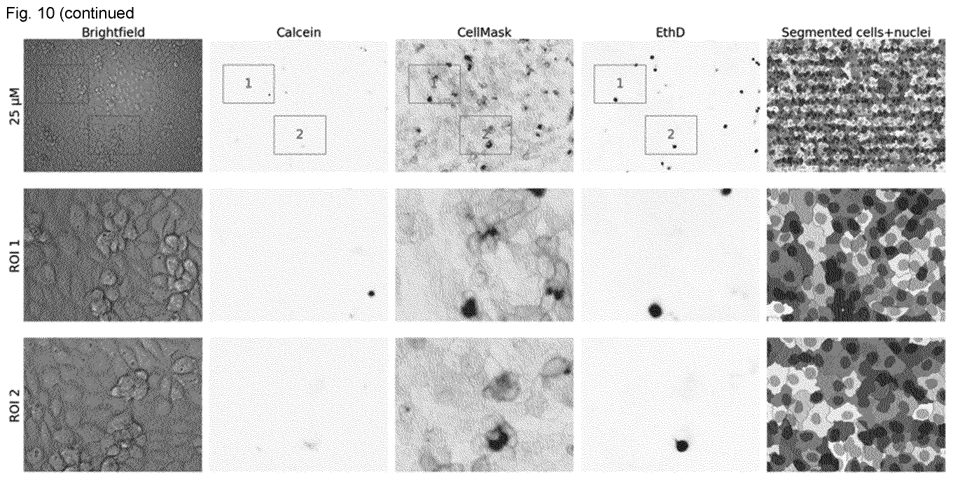

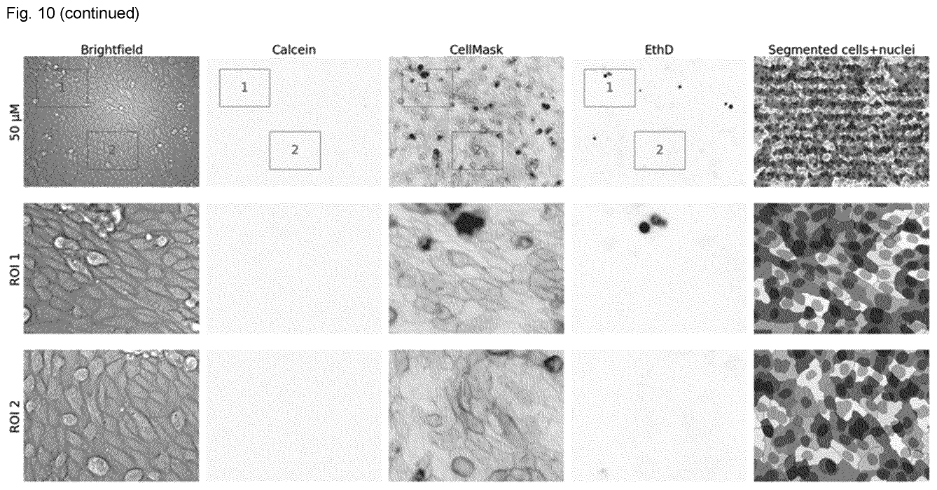

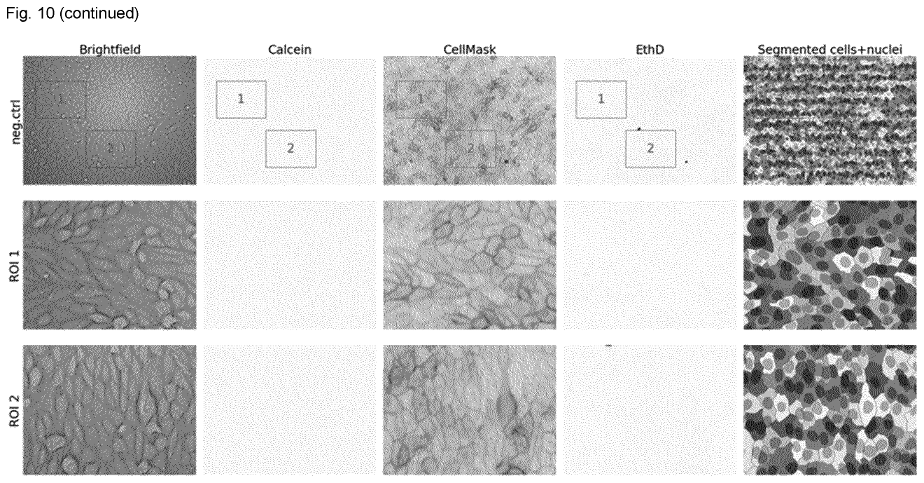

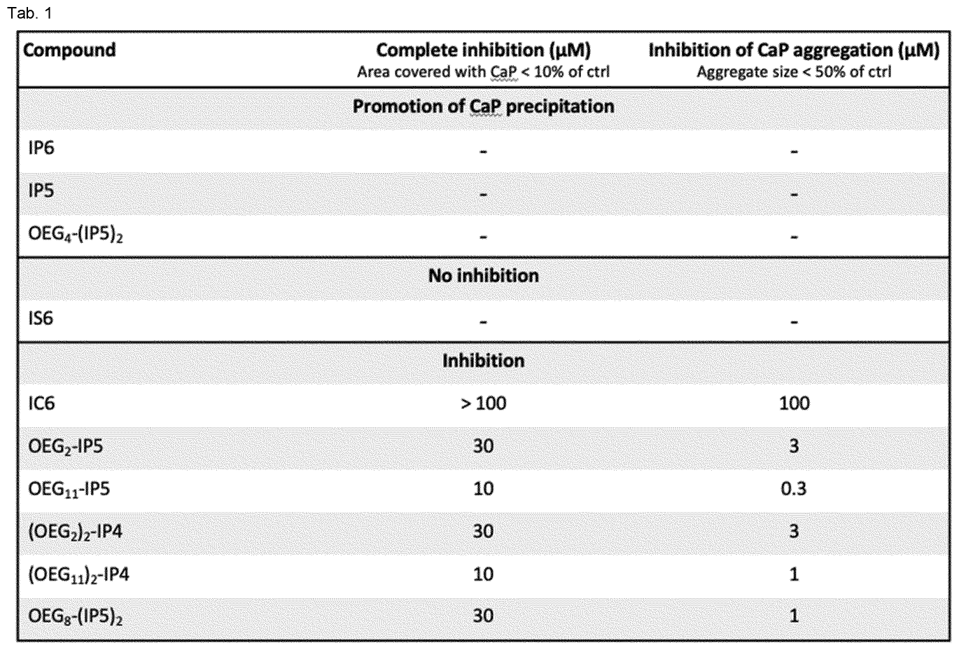

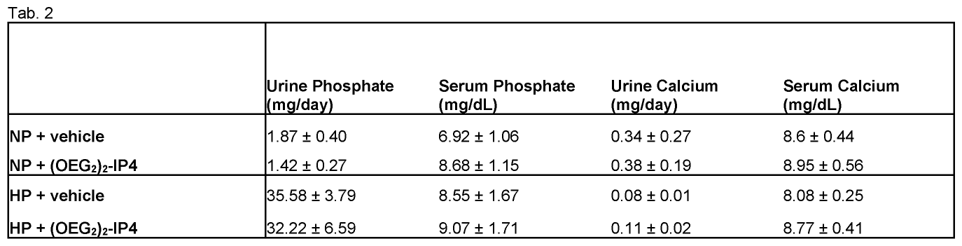

Cell medium spiked with 6 and 13 μM (OEG₂)₂-IP4 led to a reduction in total CaP area compared to the positive ctrl. It, however, still showed a high calcein staining intensity (FIG. 4C). Those results indicate that, in the positive ctrl, CaP is equally spread out across the monolayer, while with partial CaP inhibition large, localized cluster of high calcium content are formed (FIG. 4C, D, FIG. 10). The inventors assume that, as with intermediate Ca/P concentrations in the concentration range experiment, this reflects the general inhibition of CaP adhesion, except at areas of high cellular affinity for CaP. At those areas, a high accumulation of CaP is observed. As in the Ca/P concentration escalation experiments, these effects can be further supported by the SSIM of calcein and CellMask channel images. A high SSIM suggested areas of high intensity membrane stainings overlapping with high calcium regions (FIG. 4C). Additionally, the inventors observed an overlap of such areas with injured cells, as indicated by the EthD staining (FIG. 4B). Hence, such sites could present localized areas of cellular damage, and a regenerating/proliferating epithelium with surface expression of crystal binding proteins, which causes the high CaP accumulation. Increasing concentrations of (OEG₂)₂-IP4 from 6 to 13 μM first increased the correlation metric of the CellMask staining to levels above the positive ctrl, which can potentially be ascribed to a loss of cell outlines with no or limited presence of CaP-membrane clusters, before a reduction towards negative ctrl levels, wherein cells show round cellular outlines.

The inventors then tested the effects of the compound on CaP-induced CaOx crystallization. While the compound showed limited efficacy on CaOx crystallization in solution in a previous study (Kletzmayr, A. et al., Adv. Sci. 7, 1903337. DOI: 10.1002/advs.201903337, 2020), in the CaP-induced model, the inventors observed a dose-dependent change. Namely, (OEG₂)₂-IP4 could first, revert CaOx crystallization from calcium oxalate monohydrate (COM), the predominant most stable form, to calcium oxalate dihydrate (COD), in line with the inventors' former report of stepwise CaOx inhibition (Kletzmayr, A. et al., Adv. Sci. 7, 1903337. DOI: 10.1002/advs.201903337, 2020). Second, by further increasing concentrations to 100 μM COD was almost entirely abolished. Hence, (OEG₂)₂-IP4 reduced CaP-induced CaOx crystallization, which might be caused by coating and shielding of CaP deposits with compound.

Taken together, (OEG₂)₂-IP4 could prevent both CaP adhesion to the cellular monolayer and -associated cellular changes. The compound first confines CaP adhesion and cell injury to localized sites of adhesion, where CaP-membrane clusters are forming, before it completely prevents CaP deposition and cell injury at higher concentrations.

### Example 5: Ca/P Induced Transcriptomic Changes Reflect Vascular Calcification Processes In Vitro and were Prevented by (OEG2)₂-IP4

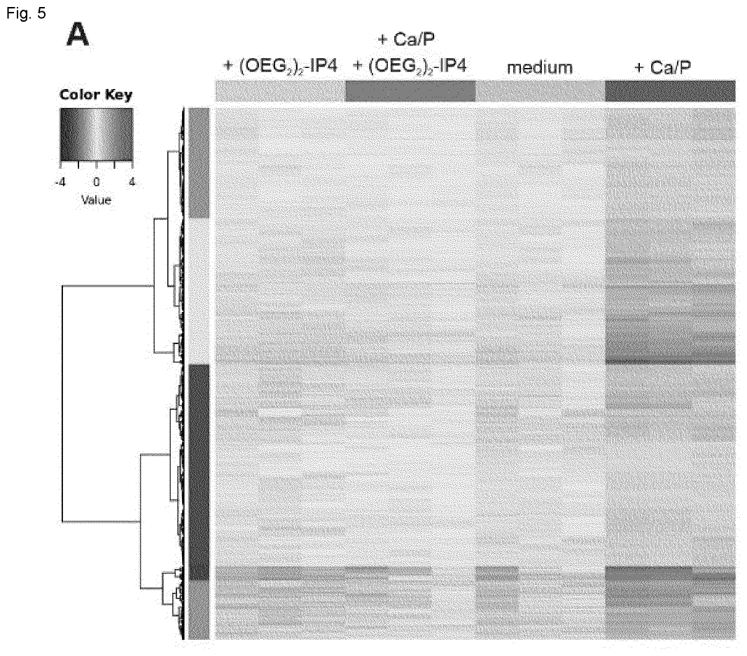

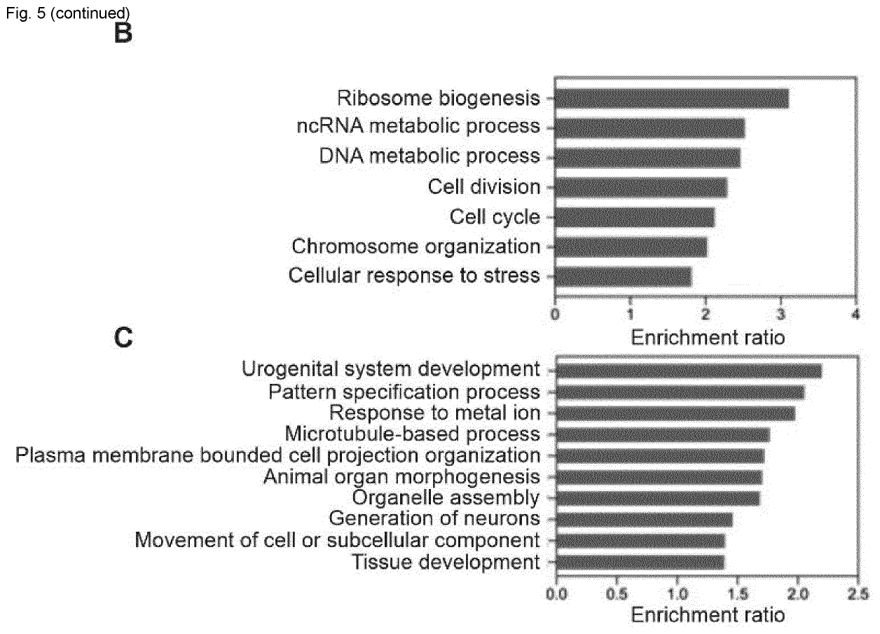

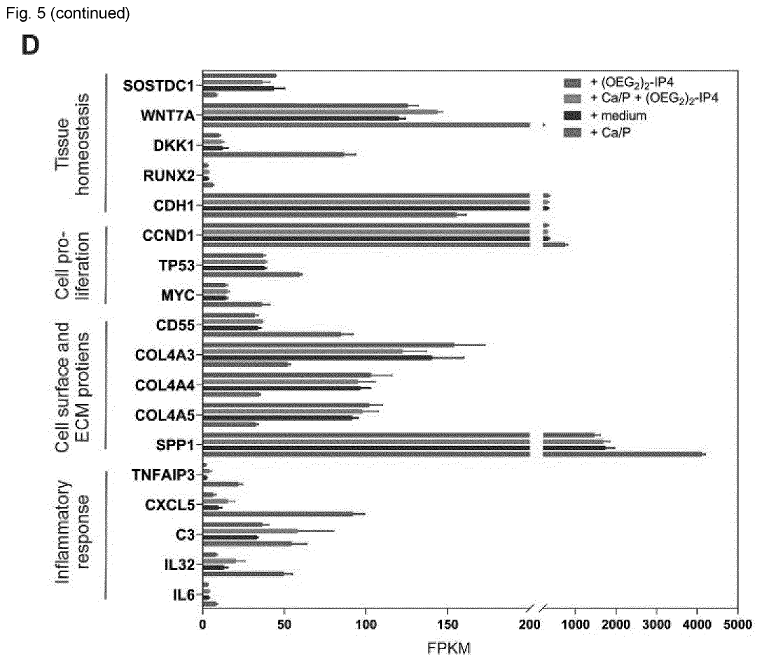

To further understand the cellular response to CaP and interpret the associated cellular morphology changes observed by imaging the inventors performed an RNA sequencing experiment. Cells were cultured as in the imaging assay and treated with Ca/P spiked medium (positive ctrl), Ca/P spiked medium premixed with 50 μM (OEG₂)₂-IP4, medium only (negative ctrl) or (OEG₂)₂-IP4 only. Hierarchical clustering of differentially expressed genes between positive and negative ctrl showed a drastic change in gene expression profile with Ca/P treatment, which was prevented by (OEG₂)₂-IP4 spiking (FIG. 5A). In positive ctrl vs. negative ctrl samples 2818 differentially expressed genes with a fold change ≥1.5 and p≤0.05 were detected, similar to Ca/P+(OEG₂)₂-IP4 and (OEG₂)₂-IP4 vs. positive ctrl samples (2437 and 2935 differentially expressed genes, respectively). In contrast, extremely limited numbers of differentially expressed genes were detected between Ca/P+(OEG₂)₂-IP4 vs. negative ctrl and vs. (OEG₂)₂-IP4 only (76 and 77, respectively). Thereby the drastic change induced by Ca/P treatment and the prevention thereof by (OEG₂)₂-IP4 was confirmed.

Overrepresentation analysis of upregulated genes in positive vs. negative ctrl samples revealed an enrichment in cell cycle, cell division and associated processes (DNA metabolic processes, ribosome biogenesis, chromosome organization), as well as cellular stress response processes (FIG. 5B). Downregulated genes enriched in structural and developmental processes (FIG. 5C).

The inventors next looked at single gene expression levels, focusing on four groups of genes, namely inflammatory response pathways, extracellular matrix (ECM) proteins, cell cycle and proliferation processes and genes involved in tissue homeostasis. Ca/P treatment of cells induced inflammatory response pathways, as reported previously for CaOx crystals (Kletzmayr, A. et al., Adv. Sci. 7, 1903337. DOI: 10.1002/advs.201903337, 2020). Upregulated genes included interleukin-6 (IL6) and interleukin-32 (IL32), complement C3 (C3), C-X-C motif chemokine ligands (e.g. CXCL5) and TNF signalling pathway genes, such as TNF alpha induced protein 3 (TNFAIP3) (FIG. 5D). Further extracellular matrix and cell surface genes were investigated. Putative calcium crystal binding proteins, such as the cell surface glycoproteins Osteopontin (SPP1) or CD55, were upregulated with Ca/P addition. In contrast, collagen IV family members (COL4A3, COL4A4, COL4A5) were downregulated. Collagen IV presents the major protein component of the tubular basement membrane. Hence, these data suggest strong alterations of the basement membrane, which might contribute to calcification of the basement membrane observed in kidney stone formers.

Overrepresentation analysis revealed a drastic deregulation of cell cycle and division processes. Upregulation of myc, a pro-proliferative gene, and cyclin D1 (CCND1), regulating the cell cycle in the G1/S transition, might indicate that renal epithelial cells enter a proliferative state upon Ca/P stimulation. This notion is further supported by the simultaneous upregulation of TP53, a regulator of DNA damage recognition and repair at the G1/S regulation point.

Furthermore, deregulation of genes involved in developmental processes and tissue homeostasis suggest changes in cellular differentiation upon Ca/P stimulation. Expression of e-cadherin (CDH1), an epithelial cell marker, was reduced upon Ca/P treatment, indicating a loss of the epithelial phenotype. The wnt signalling pathway was reported to promote osteogenic transdifferentiation of vascular cells and vascular calcification by directly modulating Runx2 gene expression. Expression of several wnt signalling pathway genes was deregulated upon Ca/P stimulation of renal epithelial cells, including Wnt family member 7A (WNT7A), sclerostin domain containing 1 (SOSTDC1) and dickkopf WNT signalling pathway inhibitor 1 (DKK1). Additionally, Runx2 expression was upregulated upon Ca/P treatment.

Therefore, RNA sequencing suggested drastic cellular alterations upon Ca/P stimulation, including a loss of the epithelial phenotype towards a more proliferative state and a change in cellular differentiation similar to vascular calcification processes. (OEG₂)₂-IP4 could largely prevent Ca/P induced changes, likely due to reduced cell-crystal interactions.

### Example 6: (OEG2)₂-IP4 Reduces High Phosphate Induced Kidney Damage In Vivo

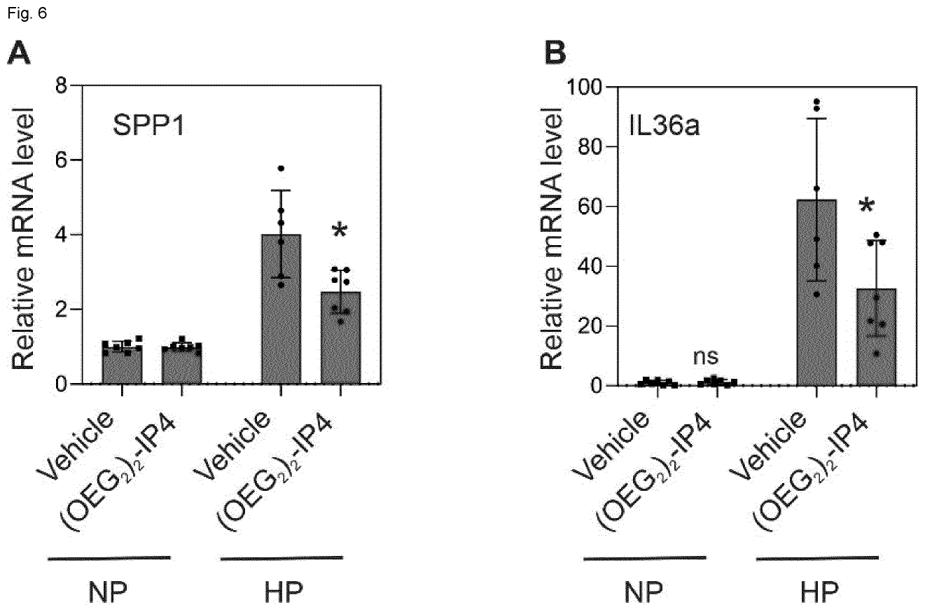

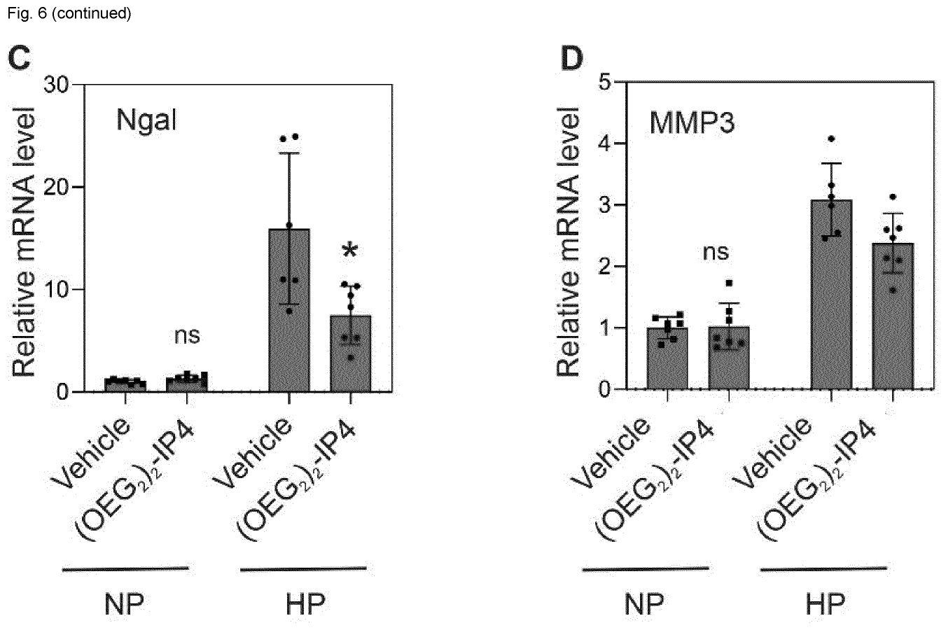

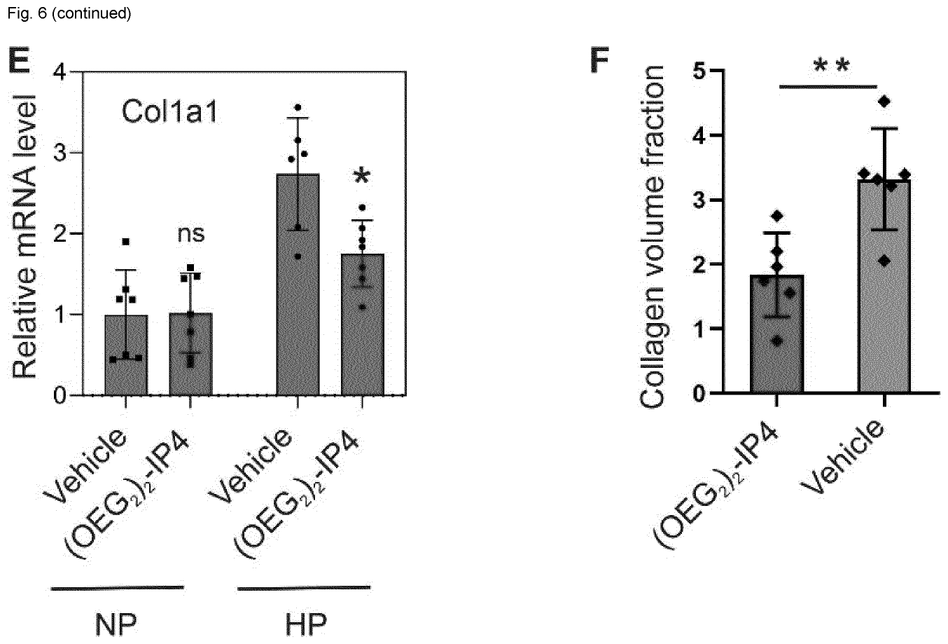

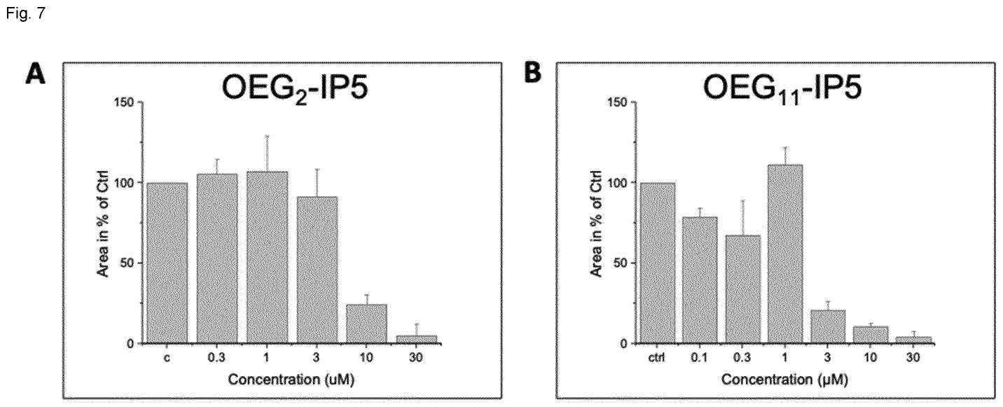

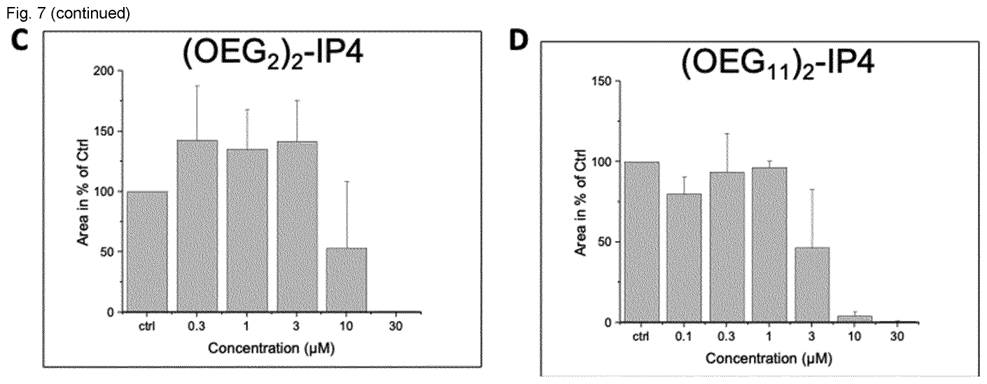

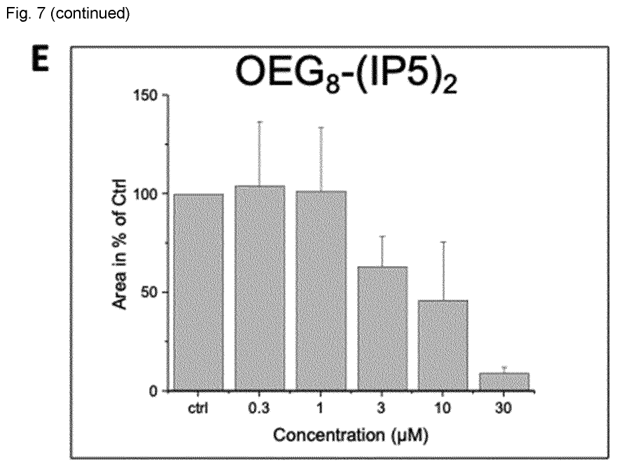

The efficacy of (OEG₂)₂-IP4 was further tested in a mouse model of high phosphate-induced kidney damage. Based on the previously performed characterization of (OEG₂)₂-IP4 pharmacokinetics in rats, a plasma concentration of roughly 80 μM after 30 min is anticipated in mice, following a subcutaneous injection of 100 mg/kg. The high phosphate diet induces FGF23 expression, compared to a normal phosphate diet, which in turn enhances renal phosphate excretion to keep serum levels within normal limits. This feedback mechanism is also suggested to contribute to high renal phosphate levels in early stage CKD. The phosphate diet induced an increase in urinary phosphate excretion from 1.9 to 35.6 mg/day, with no significant difference between the vehicle and treatment group (Table 2). Furthermore, no significant differences between the vehicle and treatment group were observed in serum phosphate, CaP or urine calcium at the end of the treatment period (Table 2). Mice on the high phosphate diet showed elevated markers of kidney damage, such as an increase in Spp1, IL-36a and Ngal (FIG. 6A-C). Further, heightened kidney expression levels of fibrosis markers, such as matrix metallopeptidase-3 (MMP) and Collagen 1a1 (Col1a1) were measured in the high phosphate vs. the normal phosphate vehicle control group. Concurrent treatment with 100 mg/kg (OEG₂)₂-IP4 3 times/week subcutaneously significantly reduced kidney damage and fibrosis markers compared to the vehicle control group in the high phosphate diet group (FIG. 6A-E). Furthermore, (OEG₂)₂-IP4 significantly reduces fibrosis, as measured by a reduced collagen volume fraction following Picro-Sirius red staining of the kidneys (FIG. 6F). Hence, the inventors' preliminary results suggest a beneficial effect of (OEG₂)₂-IP4 on phosphate-induced kidney injury in vivo.

### DISCUSSION

Renal tubules are exposed to a wide variety of metabolites at high concentrations, sometimes causing their precipitation and cellular damage. Calcium precipitation in the form of CaP and CaOx is of particular concern, due to the associated kidney calcification, tissue damage and potentially accelerated progression of CKD. Because of the wide variety of nephrotoxic environmental perturbations, the inventors first aimed at establishing a simple in vitro image-based profiling tool that could allow the rapid testing of a multitude of renal perturbations, focusing on calcification conditions, and possible inhibitory molecules.

The proposed image-based calcification profiling platform allowed for simple and fast alterations of calcification conditions, i.e. ionic conditions triggering different types of calcium crystals. The inventors implemented an automated analysis pipeline, quantifying both single cell changes, as well as CaP deposition by fluorescent staining with calcein. Advantages of using an image-based profiling approach vs. e.g. RNA sequencing of bulk cells, are the possibility to detect localized changes and its high-throughput adaptability.

The inventors demonstrated a gradual change in the feature profile of renal epithelial cell monolayers with increasing Ca/P concentration in the culture medium. A loss of the distinct cobblestone-like epithelial phenotype towards an enlarged cell shape was observed. In line, RNA sequencing confirmed a loss of the epithelial marker e-cadherin and a more proliferative state of cells stimulated with Ca/P. These findings are in agreement with literature reports suggesting dedifferentiation and cellular injury processes occur upon cell-crystal interactions. Signalling pathway alterations similar to pathological changes involved in vascular calcification, suggested a possible transdifferentiation of epithelial cells towards an osteoblast-like phenotype. Interestingly, the inventors also observed changes in CaP deposition patterns. Upon lowering Ca/P load, CaP precipitation and/or adhesion were favoured at sites of cell injury and high membrane staining. At those sites, CaP accumulated, causing further injury, cell detachment and formation of CaP-membrane clusters.

Previous studies support the idea of preferred attachment of CaP to specific crystal-binding proteins, which may be expressed mainly on dedifferentiated or regenerating renal epithelial cells. RNA sequencing confirmed an enhanced expression of crystal binding cell surface and ECM proteins, such as osteopontin. The enhanced proliferation of Ca/P stimulated cells might favour uncontrolled multi-layer growth and subsequent cell detachment, which could explain the derangements in cell membrane staining and contribute to the CaP cluster formation.

Additionally, collagen IV family members, the main components of the renal tubule basement membrane, were downregulated upon Ca/P stimulation. Calcification of the basement membrane is considered the first step of CaP plaque formation in kidney stone formers, however to date, the initial calcification process remains unclear. Hence collagen IV downregulation could provide a first insight into CaP plaque formation and suggests the utility of the calcification platform for mimicking pathophysiological processes. Further studies will be needed to clarify whether initial attachment sites are formed by the CaP load, or a certain extent of cell injury precedes and is then amplified by CaP binding. The results suggest the existence of active cellular involvement in the process of kidney calcification, thus supporting the profiling of a wide array of molecules, which could act on multiple steps of the process.

In a next step, the inventors investigated the efficacy of a library of IP6 analogues on effect on renal CaP precipitation in solution and cellular adhesion in vitro. The chosen lead compound (OEG₂)₂-IP4 dose-dependently reverted the cell feature profile towards the negative ctrl profile, inhibiting single cell changes, as well as CaP deposition. Protective effects of the compound on high Ca/P induced cellular changes were confirmed by RNA sequencing. This effect might be the result of both inhibition of CaP growth and CaP adhesion. Importantly, the protective effect of the compound translated to efficacy in a mouse model of high phosphate induced kidney damage. Hence, the inventors believe that, by inhibiting CaP precipitation and CaP-cell interactions, (OEG₂)₂-IP4 has the potential to prevent CaP-accelerated injury of the kidney. Additionally, the compound reduced CaP-induced CaOx crystallization on the cell monolayer in vitro. These results suggest a potential therapeutic benefit of the molecule in CaP-initiated kidney diseases.

1. A method for treatment or prevention of a disease associated with formation of, and/or tissue exposure to, calcium salt crystals, wherein the disease is selected from the group consisting of:
renal fibrosis, particularly when associated with calcification of renal tissue,
renal inflammation, particularly when associated with calcification of renal tissue,
nephritis,
interstitial nephritis,
glomerulonephritis,
phosphate-induced renal fibrosis,
phosphate-induced chronic kidney disease,
chronic kidney disease associated with hyperphosphatemia,
progression of chronic kidney disease,
phosphate toxicity,
hyperphosphaturia,
hyperphosphatemia, and
hyper-FGF23-emia,
the method comprising:
administering to a subject in need thereof a therapeutically effective amount of an inositol polyphosphate oligo alkyl ether compound, or its pharmaceutically acceptable salt, thereby treating or preventing the disease.

2. The method of claim 1, wherein the inositol polyphosphate oligo alkyl ether compound is described by a general formula I 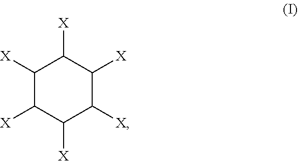
wherein
one or two or three X are oligo-ethylene glycol and the remaining X are OPO₃²⁻.

3. The method of claim 2, wherein two X are oligo-ethylene glycol and the remaining four X are OPO₃²⁻.

4. The method of claim 2, wherein three X are oligo-ethylene glycol and the remaining three X are OPO₃²⁻.

5. The method of claim 2, wherein one X is oligo-ethylene glycol and the remaining five X are OPO₃²⁻.

6. The method of claim 2, wherein the oligo-ethylene glycol is described by a formula O—(CH₂—CH₂—O)ₙCH₃, with n being selected from an integer between 2 and 20, particularly n being 2 to 12.

7. The method of claim 6, wherein n is 2.

8. The method of claim 3, wherein the compound is described by any one of the formulas:

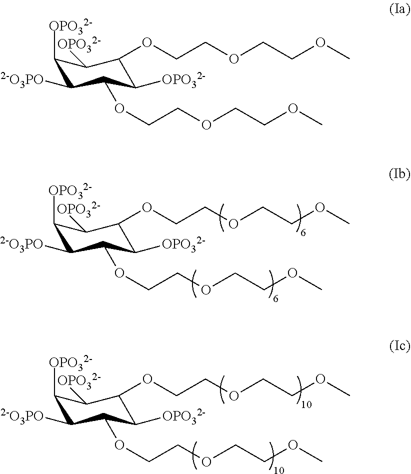

9. The method of claim 5, wherein the compound is described by any one of the formulas:

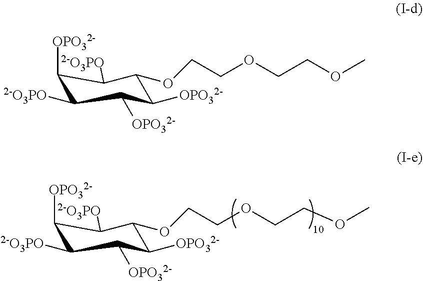

10. The method of claim 1, wherein the inositol polyphosphate oligo alkyl ether compound is described by a general formula II 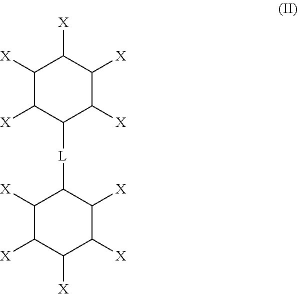
wherein
each X is OPO₃²⁻ and L is —(O—CH₂—CH₂)ₘ—O— with m having a value between 5 and 15, particularly with m having a value between 6 and 12, more particularly with m having a value between 7 and 10, even more particularly with m being 8.

11. The method of claim 10, wherein the compound is described by any one of the formulas:

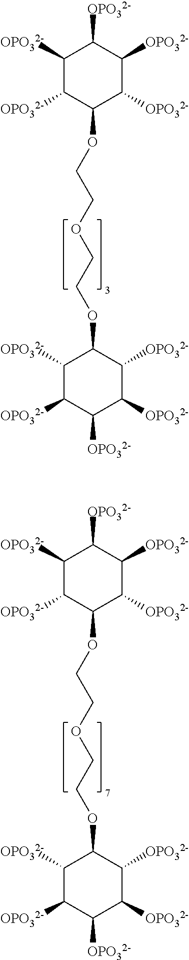

12. The method of claim 1, wherein the inositol moiety is myo-inositol.

13. The method of claim 1, wherein the disease associated is a disease associated with the formation of calcium phosphate salt or precipitate.

14. The method of claim 1, wherein the disease associated is a disease associated with the formation of calcium oxalate salt or precipitate.

15. The method of claim 1, wherein the disease associated is a disease associated with the formation of a mixed calcium phosphate oxalate precipitate.

16. The method of claim 10, wherein both inositol moieties are myo-inositol.
